(19) United States
(12) Patent Application Publication
(10) Pub. No.: US 2007/0037887 A1
(43) Pub. Date: Feb. 15, 2007

(54) Blockade of mtor to prevent a hormonal adaptive response

(75) Inventors: Richard J. Santen, Charlottesville, VA (US); Wei Yue, Charlottesville, VA (US); John C. Lawrence JR., Earlysville, VA (US); Lloyd P. McMahon, Afton, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: University of Virginia Patent Foundation, Charlottesville, VA (US)

(21) Appl. No.: 10/569,140

(22) PCT Filed: Aug. 20, 2004
(86) PCT No.: PCT/US04/27016

§ 371 Date: Feb. 21, 2006

Related U.S. Application Data
(60) Provisional application No. 60/497,067, filed on Aug. 22, 2003.

(51) Int. Cl. A61K 31/195; A61K 31/192

(52) U.S. Cl. 514/567; 514/568

(57) ABSTRACT

One embodiment of the present invention is directed to the use of an mTOR inhibitor, such as farnesylthiosalicylic acid (FTS), to prevent a hormonal adaptive response during hormonal deprivation treatment of a hormonal responsive cancer.

### RELATED APPLICATIONS

This application claims priority under 35 USC §119(e) to U.S. Provisional Application Ser. No. 60/497,067, filed Aug. 22, 2003, the disclosure of which is incorporated herein by reference.

### US GOVERNMENT RIGHTS

This invention was made with United States Government support under Grant Nos. R01 65622, P30-CA 44579, R01 84456 and R01 DK 52753, awarded by National Institutes of Health. The United States Government has certain rights in the invention.

### BACKGROUND

Clinical and biochemical data provide evidence that one-third of human breast cancers are hormone dependent. Estrogen is the predominant mitogen for these tumors and deprivation of estradiol is a common treatment for women with breast cancer. Deprivation is accomplished by removal of the ovaries, by drugs called aromatase (estrogen synthesis) inhibitors, and by use of compounds called GnRH super-agonist analogues. However, clinical observations have revealed that breast cancer cells can adapt to conditions of low estradiol by developing enhanced sensitivity to estradiol. Specifically, 200 pg/ml estradiol is required to stimulate tumor growth before acute deprivation of estradiol, whereas levels of 10-15 pg/ml are sufficient to cause tumor proliferation after adaptation 12-18 months later.

Investigations of the growth of breast cells in culture have shown that when wild-type MCF-7 cells are cultured over a prolonged period in estrogen-free medium, the cells initially stop growing but then, months later, the cells adapt and grow as rapidly as wild-type MCF-7 cells maximally stimulated with estradiol. These adapted cultured cells, named LTED (long-term estrogen deprivation) cells, have been used to study processes relating to hormone adaptation.

The MAP kinase (MAPK) pathway is believed to play an important role in stimulating the proliferation of hormone-dependent breast cancer cells, and its up-regulation has been directly measured (by detecting the level of activated MAPK) in LTED cells in vitro and in LTED xenografts in nude mice. The MAP kinase pathway stimulates the growth of cells by increasing the levels of cell cycle related proteins called cyclins. Activated MAPK is further implicated in the enhanced growth of LTED cells because inhibitors of MAPK such as PD98059 or U0126 block the incorporation of treated thymidine into DNA. These data suggest that an increase in activated MAPK participates in the adaptive hypersensitivity process.

In addition to, or in parallel with MAP kinase, the PI3 kinase pathway has also gained increasing attention as a mediator of proliferation. PI3 kinase phosphorylates and activates Akt, which can enhance cell survival, and PI3 kinase also stimulates cell proliferation through two steps involving P70-S6 kinase and 4-E-BP-1 (also called PHAS-1). In addition, Akt phosphorylates tuberous sclerosis complex 2 (TSC2), which abolishes the inhibitory effect of TSC 1/2 complex on Rheb and results in mTOR activation. mTOR, a serine/threonine protein kinase, is a central element in a signaling pathway that controls protein synthesis. p70 S6K and PHAS-I (also known as 4EBP1) are the best characterized effectors of mTOR. p70 S6K, the major ribosomal protein S6 kinase, is phosphorylated and activated by mTOR. PHAS-I binds to eIF4E, the mRNA cap binding protein and prevents the interaction of eIF4E with eIF4G, thus inhibiting cap-dependent translation. When phosphorylated in an mTOR-dependent manner in response to growth factor stimulation, PHAS-I dissociates from eIF4E, resulting in an increase in cap-dependent translation.

RAS is a key modulator of both the MAP kinase and PI3 kinase pathways and thus has been the focus of drug development for the treatment of cancers. Several studies have examined a compound, farnesylthiosalicylic acid (FTS), that inhibits binding of GTP-Ras to a plasma membrane acceptor protein, galectin 1, leading to an increase in Ras degradation. In its development, FTS has been tested in vitro and in vivo for the treatment of tumors which contain activating RAS mutations. These include malignant melanoma and pancreatic cancer. Studies have shown blockade of cell growth in tissue culture in vitro as well as inhibition of tumor growth in vivo. The agent is non-toxic and does not cause a reduction of animal weight at the doses needed to inhibit tumor growth.

One aspect of the present invention relates to the use of FTS to treat breast cancer and to prevent development of resistance to hormonal therapy. More particularly, applicants have discovered that FTS has a previously unrecognized ability to block the activity of mTOR, and the recognition of this activity has lead to the presently proposed use of FTS to inhibit the adaptive response to hormonal therapy of hormone responsive cancers.

### SUMMARY OF VARIOUS EMBODIMENTS OF THE INVENTION

One aspect of the present invention relates to the use of compounds having the general structure:

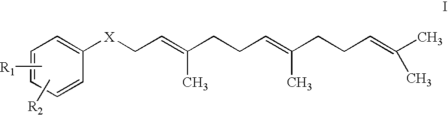

wherein X is NH, O, SO, SO₂ or S; R₁ is H or halo; and R₂ is COOH, as potent blockers of the mammalian enzyme mTOR. Compositions comprising such mTOR inhibitors can be used in one embodiment as a drug to treat breast cancer and other hormone responsive cancers. Breast cancer is a hormone dependent tumor that initially responds to blockade of estrogen synthesis or action but then later develops resistance to such a therapeutic strategy. The development of hormonal resistance is believed to involves up-regulation of mTOR mediated events, and applicants propose herein that FTS and other inhibitors of mTOR activity will provide an important and highly effective means of preventing resistance to hormone deprivation therapy.

### BRIEF DESCRIPTION OF THE DRAWINGS

FIG. 1 shows the inhibitory effect of FTS on the phosphorylation in vitro of the mTOR substrate, PHAS-I, by mTOR immunoprecipitated from cultured cells. 293T cells were transfected with expression vectors for AU1-epitope tagged mTOR, HA-tagged raptor, and HA-tagged mLST8. After 24 h, the overexpressed mTOR was immunoprecipitated with AU1 antibodies, and immune complexes were incubated with increasing concentrations of FTS in a reaction mixture containing 2 mM [γ³²P] ATP (250 μCi/ml), 20 mM MnCl₂, and 0.1 mg/ml purified recombinant histidine-tagged PHAS-1. After fifteen minutes the reaction was stopped by adding SDS sample buffer, and samples were subjected to SDS-PAGE. The amount of ³²P incorporated into PHAS-I was determined and expressed as a percentage of that incorporated in the absence of FTS. Mean values±the half the range of two experiments are presented.

FIG. 2 is a schematic representation of the signal transduction pathways involved in estrogen-induced proliferation of breast cancer cells. Arrows with a solid line indicate stimulation. Arrows with a dashed line indicate stimulation under certain circumstances. Bars indicate inhibition.

FIG. 3 represents the effect of FTS on the growth of MCF-7, LTED, and MCF-10A cells. Sixty thousand cells were plated into each well of 6-well plates. Two days later, the cells were treated in triplicate for five days with FTS at indicated concentrations. The results (mean±S.E.) are expressed as percentage of the vehicle control.

FIG. 4 represents the effect of FTS on E₂ stimulated growth of MCF-7 and LTED cells. Thirty thousand cells were plated into each well of 6-well plates. Two days later, the cells were re-fed with IMEM containing 5% DCC-FBS. After another two days, cells were treated in triplicate with estradiol (10⁻¹⁰ M) plus various concentrations of FTS for five days. In LTED cells, fulvestrant (10⁻⁹ M) was included in all wells to block the effect of residual estrogen in the culture. The results (mean±S.E.) are expressed as percentage of cell number from the wells containing E₂.

FIGS. 5A & 5B represent the effect of FTS on DNA synthesis in MCF-7 (FIG. 5A) and LTED (FIG. 5B) cells. One hundred thousand cells were plated into each well of 24-well plates. Two days later, the cells were re-fed with IMEM containing 5% DCC-FBS. After another two days, cells were treated in quadruplicate with the compounds indicated for 18 hours. [³H] thymidine (1 μCi/well) was added during the last 2 h of incubation. [³H] thymidine incorporated into DNA was normalized by protein content.

FIG. 6 is a bar graph portraying data representing the induction of apoptosis in MCF-7 and LTED cells by FTS. Eighty thousand MCF-7 and LTED cells were plated into each well of 12-well plates in their culture media. Two days later, the cells were treated with FTS for 3 days. Apoptosis was measured using a Cell Death Detection Kit from Roche Molecular Biochemicals. Parallel plates subjected to identical treatment were prepared for cell counting. The results were expressed as the values of optical density at 405 nm per 10,000 cells.

FIGS. 7A-7D represent data showing the effect of FTS on serum-stimulated activation of MAP kinase, PI3 kinase, and mTOR. Subconfluent MCF-7 and LTED cells grown in 60 mm dishes were treated with FTS in their culture media for 24 h. Cells were then harvested and cell lysate prepared. Phosphorylation of ERK1/2 MAP kinase (FIG. 7A), Akt at Ser⁴⁷³ (FIG. 7B), p70 S6 kinase at Thr³⁸⁹ (FIG. 7C), and PHAS-I at Ser⁶⁵ (FIG. 7D) was detected by Western analysis using specific antibodies and quantitated by densitometry scanning.

FIG. 8 represents data showing the effect of FTS on EGF induced activation of MAP kinase, PI3 kinase, and mTOR in LTED cells. Subconfluent LTED cells grown in 60 mm dishes were serum starved for 24 h, pretreated for 1 h with FTS, 3 h with LY 294002 (LY), or rapamycin (Rapa) at indicated concentrations before addition of EGF (1 μg/ml, 1 h). Cells were then harvested and cell lysate prepared. Phosphorylated and total kinases were detected by Western analysis using specific antibodies.

FIG. 9. represents data showing the effect of FTS on IGF-1 induced activation of MAP kinase, PI3 kinase, and mTOR in LTED cells. Subconfluent LTED cells grown in 60 mm dishes were serum starved for 24 h, pretreated with FTS (100 μM) or LY 294002 (LY, 20 μM) for 10, 30 or 60 min before addition of IGF-1 (20 ng/ml for 10 min). Cells were then harvested and cell lysates prepared. Phosphorylated and total kinases were detected by Western analysis using specific antibodies.

FIGS. 10A-10D represent data showing the effect of FTS on serum- and growth factor-induced phosphorylation of p70 S6 kinase at Thr²²⁹ in LTED cells (FIG. 10A). Comparison of FTS and LY 294002 (LY) on Thr²²⁹ phosphorylation of p70 S6K induced by EGF (FIG. 10B; by same treatment as described in FIG. 8). Comparison of FTS and LY 294002 on Thr¹²⁹ phosphorylation of p70 S6K induced by IGF-1 (FIG. 10C; by same treatment as described in FIG. 8). FIG. 10D is graph displaying the time course of FTS (100 μM) effects on Thr³⁸⁹ and Thr²²⁹ phosphorylation of p70 S6K in LTED cells cultured in serum containing IMEM.

FIG. 11A & 11B represent data showing FTS inhibits PHAS-I phosphorylation and promotes dissociation of the mTOR-raptor complex in 293T cells. 293T cells were transfected with pcDNA3_{AU1-mTOR}, pcDNA3_{3HARaptor}, pcDNA3_{3HA-mLST8}, and pCMV-Tag3A_{PHAS-I} (FIG. 11A). After 18 h the cells were incubated for 1 h with increasing concentrations of FTS before extracts were prepared. A sample of each extract was subjected to SDS-PAGE and immunoblotted to detect PHAS-I or PThr^{36/45}. AU1-mTOR was also immunoprecipitated from each extract, and immune complexes were subjected to SDSPAGE and immunoblotted with mTAb2, to detect mTOR, and with anti-HA antibodies, to detect HA-mLST8 and HA-raptor. Nontransfected 293T cells were incubated with increasing concentrations of FTS for 1 h before extracts were prepared. mTOR was immunoprecipitated with mTAb1, and samples were subjected to SDS-PAGE. Immunoblots of mLST8, mTOR, and are presented in FIG. 11B.

FIGS. 12A & 12B represent data showing the effects of incubating cells with increasing concentrations of FTS and GTS on mTOR activity and mTOR association with raptor and mLST8. AU1-mTOR, HA-raptor, and HAmLST8 were overexpressed in 293T cells. The cells were then incubated for 1 h with increasing concentrations of FTS (●, ▴, ▪) or GTS (∘, Δ, □) before extracts were prepared. Immunoprecipitations were then conducted with anti-AU1 antibodies. mTOR kinase activity (●, ∘) was determined by measuring ³²P incorporation into [His⁶]PHAS-I in immune complex kinases assays performed with [γ-³²P]ATP. The relative amounts of HA-raptor (▴, Δ) and HA-mLST8 (▪, □) that coimmunoprecipitated with AU1-mTOR were determined after immunoblotting with anti-HA antibodies. The results (mean values±S.E. from 3 experiments) are expressed as percentages of the mTOR activity (FIG. 12A) or coimmunoprecipitating proteins (FIG. 12B) from samples incubated without FTS or GTS, and have been corrected for the amounts of AU1-mTOR immunoprecipitated.

FIGS. 13A & 13B represent data showing FTS promotes raptor dissociation and inhibits mTOR activity in cell extracts. 293T cells were transfected with pcDNA3 alone (Vec.) or with a combination of pcDNA3_{AU1-mTOR}, pcDNA3_{3HA-Raptor}, and pcDNA3_{3HA-mLST8}. Extracts of the cells were incubated with increasing concentrations of FTS before AU1-mTOR was immunoprecipated. Samples of the immune complexes were incubated with [γ-³²P]ATP and recombinant [His⁶]PHAS-I, then subjected to SDS-PAGE and transferred to an immobilon membrane. After obtaining a phosphorimage of the membrane to detect ³²P-PHAS-I, the membrane was immunoblotted with PThr^{36/45} antibodies. Other samples of the immune complexes were subjected to SDS-PAGE and immunoblots were prepared with antibodies to the HA epitope or to mTOR (see FIG. 13A). Extracts of nontransfected 293T cells were incubated with increasing concentrations of FTS before mTOR was immunoprecipated with mTAb1. A control immunoprecipitation was conducted using nonimmune IgG (NI). Immune complexes were subjected to SDS-PAGE and immunoblots were prepared with antibodies to mLST8, mTOR, and raptor.

FIGS. 14A & 14B represent data showing the relative effects of increasing concentrations of FTS and GTS on mTOR activity and the association of mTOR and raptor. Samples of extracts from 293T cells overexpressing AU1-mTOR, HA-raptor, and HA-mLST8 were incubated for 1 h with increasing concentrations of FTS (●, ▴, ▪) or GTS (∘, Δ, □) before immunoprecipitations were conducted with anti-AU1 antibodies. mTOR kinase activity (●, ∘) was determined by measuring ³²P incorporation into [His⁶]PHAS-I in immune complex kinases assays performed with [γ-³²P]ATP (see FIG. 14A). The relative amounts of HA-raptor (▴, Δ) and HA-mLST8 (▪, □) that coimmunoprecipitated with AU1-mTOR were determined after immunoblotting with anti-HA antibodies (see FIG. 14B). The results (mean values+S.E. from 5 experiments) are expressed as percentages of the mTOR activity (FIG. 14A) or coimmunoprecipitating proteins (FIG. 14B) from samples incubated without FTS or GTS, and have been corrected for the amounts of AU1-mTOR immunoprecipitated.

FIGS. 15A-15C represent data showing the effect of mTOR inhibitors on the association of mTOR and raptor. 293T cells were transfected with pcDNA3 alone (Vec.) or with a combination of pcDNA3_{AU1-mTOR}, pcDNA3_{3HARaptor}, and pcDNA3_{3HA-mLST8}. AU1-mTOR was immunoprecipitated and samples of the washed immune complexes were at incubated at 300 for 30 min with no inhibitors or with the following: caffeine (1 mM), FTS (50 μM), LY294002 (10 μM), rapamycin (1 μM) plus FKBP12 (10 μM), and 1 μM wortmannin. To prevent destruction of wortmannin, incubations were conducted in the absence of thiol reducing agents. Samples of the immune complexes were incubated for 10 min with 10 mM MnCl₂, 1 mM [γ-³²P]ATP, and 50 μg/ml [His⁶]PHAS-I to assess mTOR activity. Other samples were rinsed twice, subjected to SDS-PAGE, and immunoblotted with anti-HA and anti-AU1 antibodies to determine the amounts of HA-raptor and HA-mLST8 associated with AU1-mTOR. A phosphorimage of ³²P-PHAS-I from the kinase assay and immunoblots of HA-mLST8, AU1-mTOR and HA-raptor are shown in FIG. 15A). The relative amounts of ³²P incorporated into PHAS-I were determined by phosphorimaging (see FIG. 15B). The amounts of HAraptor and HA-mLST8 that coimmunoprecipitated with AU1-mTOR were determined from immunoblots (see FIG. 15C). In FIG. 15B and FIG. 15C, the results were corrected for the amounts of AU1-mTOR immunoprecipitated and are expressed as percentages of the respective controls. Means+½ the range from 2 experiments are presented.

FIG. 16 represents data showing the in vivo effect of FTS on LTED cells implanted in mice. Female CrtCD1 nude mice, 3-4 weeks old, were ovariectomized and inoculated with LTED cells (5 million per site, s.c.) on both flanks of the body. Estradiol containing silastic capsule was implanted subcutaneously to provide plasma estradiol concentration approximately 10 pg/ml. Two weeks after cell inoculation, animal were divided into three groups. Animals received daily injection (ip) of phosphate-buffered saline (Buffer), cyclodextrin (CD), or FTS-CD (40 mg/kg), respectively. After seven weeks of treatment, animals were sacrificed and tumors weighed.

FIGS. 17A & 17B represent data showing the effects of FTS on JNK activation in LETD cells cultured in vitro. As shown in FIG. 17A, administration of FTS stimulates JNK activation in LETD cells cultured in vitro. Furthermore, as shown in FIG. 17B, FTS and estradiol administration to LTED cells both increase activation of JNK and result in enhances phosphorylation of cJUN.

### DETAILED DESCRIPTION OF EMBODIMENTS

Definitions

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells or reducing the size of a tumor. Additional signs of successful treatment of cancer include normalization of tests such as white blood cell count, red blood cell count, platelet count, erythrocyte sedimentation rate, and various enzyme levels such as transaminases and hydrogenases. Additionally, the clinician may observe a decrease in a detectable tumor marker such as prostatic specific antigen (PSA).

As used herein, the term “hormone deprivation therapy” relates to any treatment of a patient that blocks the action of, or removes (either by preventing synthesis or enhancing the destruction of the hormone) the presence of hormones, from a patient.

As used herein, the term “hormone responsive cells/tissue” relates to non-cancerous cells or tissues that are naturally responsive to estrogens or androgens, wherein the cells or tissue proliferate and/or initiate new protein synthesis in the presence of the hormone. Hormone responsive tissues include the mammary glands, testes, prostate, uterus and cervix. A tissue which is normally responsive to estrogens or androgens may lose its responsiveness to the hormone. Thus, “hormone responsive tissue” is a broad term as used herein and encompasses both hormone-sensitive and hormone insensitive tissues that are normally responsive to hormones. An “estrogen responsive cell/tissue” is one that is responsive to estrogen.

As used herein, the term “hormone responsive cancers” relates to cells or tissues that are derived from hormone responsive cells/tissue, and an “adapted hormone responsive cancer cell” is a hormone responsive cancer cell that will proliferate in response to levels of hormone that would not produce a response in a corresponding hormone responsive cell.

As used herein, the term “adapted hormone response” or “adapted response” relates to the process by which cells or tissues that are derived from hormone responsive tissue become able to respond to (i.e. proliferate and/or initiate new protein synthesis) levels of hormone that previously would not produce a response in those cells.

As used herein, the term “inhibition of mTOR activity” relates to a detectable decrease in mTOR's ability to phosphorylate one or more of its substrates including, for example, p70 S6K and PHAS-I. An mTOR inhibitor is a compound that has a direct inhibitory effect on mTOR activity (i.e. the inhibition of mTOR activity is not mediated though an inhibitory effect on an upstream pathway enzyme).

As used herein, the term “estrogen” relates to a class of compounds including naturally occurring and synthetically made compositions that have a demonstrated ability to induce cell proliferation and/or initiate new protein synthesis in estrogen responsive cells. Naturally occuring estrogens include estrone (E1), estradiol-17B (E2), and estriol (E3), and of these, estradiol is the most active pharmacologically.

Synthetic estrogens are compounds that do not occur in nature and duplicate or mimic the activity of endogenous estrogens in some degree. These compounds include a variety of steroidal and non-steroidal compositions examplified by dienestrol, benzestrol, hexestrol, methestrol, diethylstilbestrol (DES), quinestrol (Estrovis), chlorotrianisene (Tace), and methallenestril (Vallestril).

As used herein, the term “estrogen antagonist” relates to a compound that has a neutralizing or inhibitory effect on an estrogen's activity when administered simultaneously with that estrogen. Examples of estrogen inhibitors include tamoxifen and toremifene.

As used herein, the term “aromatase inhibitor” relates to a composition that block the conversion of androstenedione to estrone and/or testosterone to estradiol. Aromatase inhibitors include both steroidal and nonsteroidal classes of inhibitors including for example, exemestane, anastrozole and letrozole.

As used herein, the term “breast cancer” relates to any of various carcinomas of the breast or mammary tissue.

### Embodiments

Hormone dependent breast cancer in women responds to deprivation of female hormone levels with a cessation of growth of the tumor cells and with an increase in the rate of cell death. Women experience regression of tumor growth while receiving hormone deprivation therapy for about 12-18 months but thereafter, the tumor develops resistance to this therapy and tumor cell proliferation is reinitiated. Prevention of the adaptive process that leads to resistance to hormone deprivation therapy could potentially result in enhanced duration and efficacy of the endocrine deprivation therapy.

Applicants' studies of the adaptive response mechanisms that allow breast cancer cells to resume their growth after an initial response to hormone deprivation therapy have demonstrated that breast cancer cells adapt under the pressure exerted by estradiol deprivation by up-regulating two key pathways, one regulated by MAP kinase and the other by PI-3-kinase. The MAP kinase pathway stimulates the growth of cells by increasing the levels of cell cycle related proteins called cyclins. The PI-3 kinase pathway prevents cell death by activating AKT, and stimulates cell proliferation through two steps involving P70-S6 kinase and 4-E-BP-1 which is also called PHAS-1.

Applicants have postulated that blocking the two pathways that are upregulated during estradiol deprivation will prevent the development of hormonal resistance. As shown herein, the blockade of these two pathways markedly reduces the sensitivity of cells to the effects of estradiol on cell proliferation. Once the enzymatic pathways relevant to the adaptive response have been identified a determination must be made as to whether it would be more efficacious to block upstream or downstream events in the adaptive processes. One strategic approach would be to block upstream growth factors in pathways involving HER1, -2, -3, or -4; the IGF-I and II receptors; or the fibroblast growth factor family for each of these receptors. An alternative approach is to block the adaptive process at a downstream step involving MAPK and PI3K or even further downstream processes. In accordance with one embodiment of the present invention a composition and method is provided for blocking the PI-3 and MAP kinase pathways as a treatment for hormone responsive cancers.

Long-term estradiol deprivation causes an up-regulation of the amount of ER present and of processes involved in utilization of membrane-related ER. This results in an increased level of activation of the MAPK as well as the PI3K pathways. All of these signals converge on downstream pathways directly involved in cell cycle functionality and probably exert synergistic effects at that level. As a reflection of this, E2F1, an integrator of cell cycle-stimulatory and -inhibitory events, is hypersensitive to the effects of estradiol in LTED cells. Applicants believe that hypersensitivity reflects a downstream synergistic interaction of several pathways converging at the level of the cell cycle. An increase in the basal level of transcription of ER-regulated genes may also be involved in the process but does not represent the proximate cause of hypersensitivity, because transcriptional events respond to estradiol with similar dose-response curves in wild-type and LTED cells.

Because of the key role of RAS in both of the MAP and PI3 kinase pathways, initial efforts were focused upon the blockade of RAS as a key target for drug development for preventing hormonal therapy resistance in breast cancer. Farnesylthiosalicylic acid (FTS) is a known antagonist of Ras, causing displacement of the binding of RAS to an acceptor protein located in the cell membrane, leading to subsequent re-entry into the cytoplasm of the cell and degradation. See U.S. Pat. No. 6,462,086, the disclosure of which is incorporated herein in its entirety.

FTS has been tested in vitro and in vivo for the treatment of tumors which contain activating RAS mutations. These include malignant melanoma and pancreatic cancer. Studies have included blockade of cell growth in tissue culture in vitro as well as inhibition of tumor growth in vivo. The agent is non-toxic and does not cause a reduction of animal weight at the doses needed to inhibit tumor growth. However, applicants have discovered that despite its antagonist activity against Ras, surprisingly, FTS exhibited relatively weak blocking effects on MAP kinase activation.

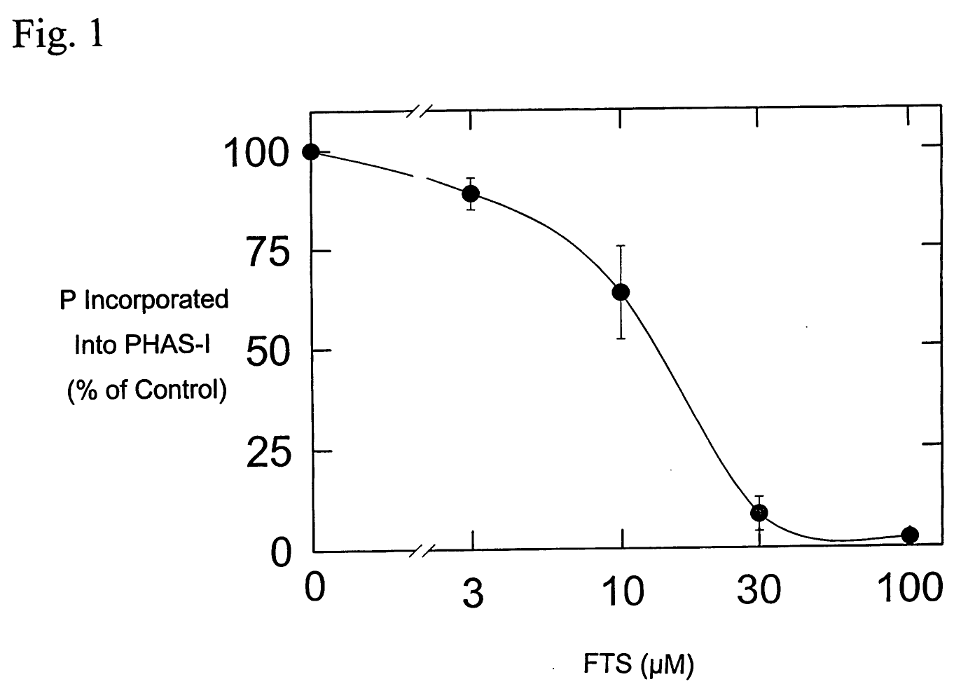

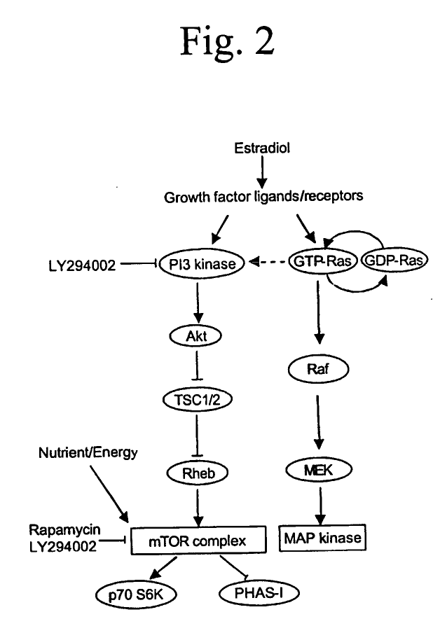

Applicants did observe that FTS markedly inhibited the phosphorylation of PHAS-I and p70 S6 kinase, two effectors of mTOR (a Ser/Thr protein kinase involved in the control of cell growth and proliferation; See FIG. 1). These results indicate that FTS is a potent inhibitor of mTOR signaling. This previously unrecognized activity of FTS could be potentially more important than its anti-RAS effect and has led to the proposed new uses of FTS to treat hormone responsive cancers. As described in Examples 4 and 5, FTS inhibits mTOR by promoting dissociation of an important subunit of a functional mTOR signaling complex. The results of these studies indicate that the mTOR pathway is a key component of cell proliferation in hormone dependent cancers, including breast cancer, and that FTS is a promising agent to interrupt mTOR signaling in hormone dependent cancers.

In accordance with one embodiment of the present invention a method of inhibiting mTOR activity in a cell is provided. The method comprises the step of contacting the cell with a composition comprising a compound of the general structure:

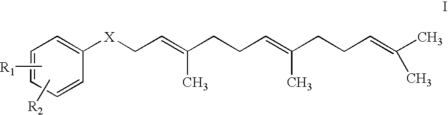

wherein X is NH, O or S; R₁ is H or halo; and R₂ is COOH. In one embodiment, the composition comprises the compound of Formula I wherein X is NH or S; R₁ is H or halo; and R₂ is COOH. In another embodiment the composition comprises the compound of Formula I wherein X is O; R₁ is H or halo; and R₂ is COOH. In another embodiment the composition comprises the compound of Formula I wherein X is S; R₁ is H or halo; and R₂ is COOH. In another embodiment the composition comprises the compound of Formula I wherein X is NH; R₁ is H or halo; and R₂ is COOH. In another embodiment the composition comprises the compound of Formula I wherein X is S; R₁ is H or F; and R₂ is COOH. In another embodiment the composition comprises the compound of Formula I wherein X is O; R₁ is H or F; and R₂ is COOH. In another embodiment the composition comprises the compound of Formula I wherein X is S; R₁ is H; and R₂ is COOH, wherein the inhibitory effect results from the dissociation of raptor from mTOR.

In accordance with one embodiment compounds suitable for use in accordance with the present invention include compounds having the following structure:

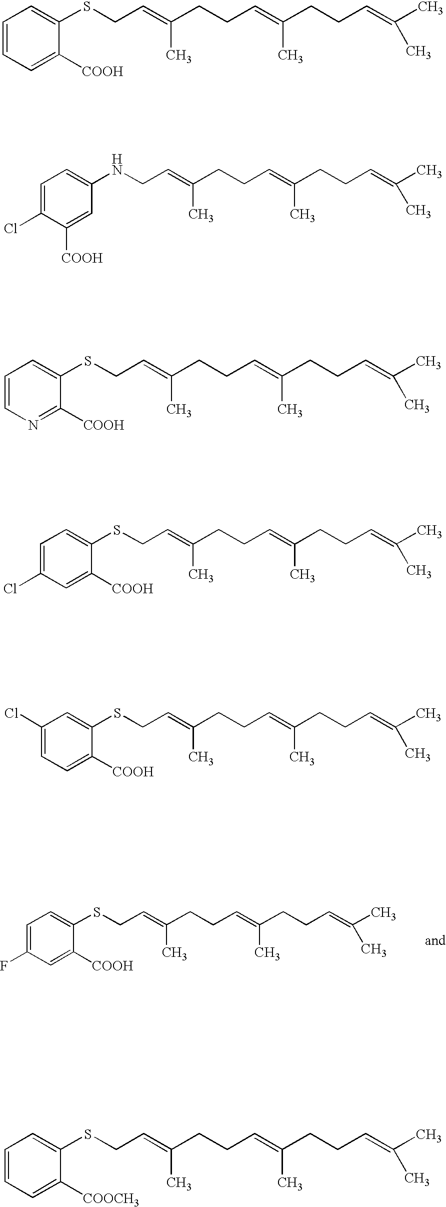

In one embodiment of the present invention a method of preventing or slowing the occurrence of the adaptive response that accompanies hormone deprivation therapy of hormone responsive cancers comprises the administration of an mTOR inhibitor. In one embodiment the mTOR inhibitor comprises one or more of the compounds described in U.S. Pat. No. 5,507,528, the disclosure of which is incorporated herein. In one embodiment the mTOR inhibitor has the general structure:

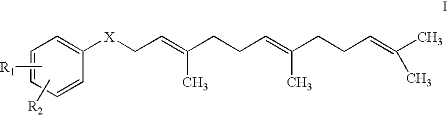

wherein X is NH or S; R₁ is H or halo; and R₂ is COOH. In another embodiment the mTOR inhibitor has the general structure of formula I wherein R₁ is H, R₂ is COOH and X is S. In another embodiment the mTOR inhibitor is FTS, the structure of which is shown below: 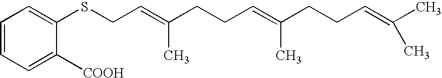
The mTOR inhibitory compounds of the present invention can be combined with pharmaceutically acceptable carriers to prepare compositions for administration to warm blooded vertebrates, including humans and other mammals to treat hormone responsive malignancies.

In accordance with one embodiment an mTOR inhibitor (such as FTS) is administered in conjunction with hormone deprivation therapy to treat a hormone responsive cancer. Use of the mTOR inhibitor “in conjunction” with hormone deprivation therapy is intended to encompass all therapeutic regimens wherein during the course of a hormone deprivation treatment an mTOR inhibitor is administered. In one embodiment the mTOR inhibitor is administered simultaneously with hormone deprivation, either as a single composition or two separate compositions that are administered sequentially one after the other. Alternatively, the mTOR inhibitor can be administered at a predetermined interval after administration of the initial dose of the hormone deprivation composition, including 1, 3, 5, 7 hours or 1, 2, 3, 4 weeks or 1, 2, 3, 4, 6, 12, 18 months after the initial administration of the hormone deprivation therapy. In accordance with one embodiment the mTOR inhibitor can be administered every time the patient receives a dosage of the hormone deprivation therapy. Alternatively, in one embodiment the mTOR inhibitor is administered at a frequency less than the frequency of hormone deprivation therapy administration.

In one embodiment the hormone responsive cancer is one that is responsive to estrogen, or a compound that displays estrogen activity. More particularly, in one embodiment an mTOR inhibitor is administered to treat cervical, ovarian or breast cancers, and in one embodiment the cancer to be treated is breast cancer. In one embodiment the mTOR inhibitor is used in conjunction with an estrogen antagonist, such as tamoxifen or toremifene. In another embodiment the mTOR inhibitor is used in conjunction with an aromatase inhibitor, including compounds selected from both the steroidal and nonsteroidal classes of inhibitors. For example, the aromatase inhibitor may be selected from the group consisting of exemestane, anastrozole and letrozole. In another embodiment the hormone responsive cancer to be treated is prostate cancer.

In one embodiment of the present invention a composition is provided for treating a hormone responsive cancer such as breast cancer, wherein the composition comprises an mTOR inhibitor and an estrogen derivation composition comprising a compound selected from the group consisting of estrogen antagonists and aromatase inhibitors. In one embodiment the composition comprises an mTOR inhibitor of the general formula:

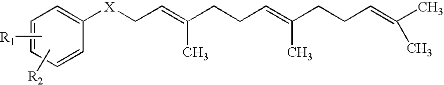

wherein X is NH or S; R₁ is H or halo; and R₂ is COOH and a compound selected from the group of estrogen antagonists or aromatase inhibitors. In accordance with one embodiment the estotrogen antagonist is tamoxifen. In one embodiment the aromatase inhibitor is selected from the group consisting of exemestane, formestane, aminoglutethimide, anastrozole and letrozole. In another embodiment the composition comprises FTS and an estrogen antagonist or aromatase inhibitor, and in another embodiment the composition comprises FTS and tamoxifen.

Compositions comprising an mTOR inhibitor can also be used in accordance with the present invention to prevent or slow the progression of the adaptive response to hormone deprivation therapy. In accordance with one embodiment, the mTOR inhibitor is administered prior to, simultaneously or after administration of the first hormone deprivation therapy dose. In one embodiment a composition comprising a compound of the formula:

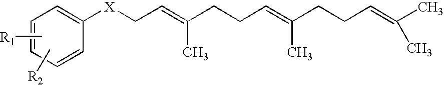

wherein X is NH or S; R₁ is H or halo; and R₂ is COOH is administered to a patient undergoing estrogen deprivation therapy to prevent or slow the progression of an adapted response.

The mTOR inhibiting compositions of the present invention can also be used in accordance with one embodiment to inhibit the growth of a partially or fully adapted hormone responsive cancer cell. A partially adapted hormone responsive cell is one that is capable of undergoing further adaptation and thus becoming capable of responding to even lower amounts of hormone than that to which the cell currently shows a response. The method of inhibiting the growth of a partially or fully adapted hormone responsive cancer cell comprises the step of administering an mTOR inhibitor compound to a patient undergoing hormone deprivation therapy. In accordance with one embodiment the mTOR inhibitor has the general formula:

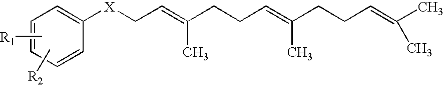

wherein X is NH or S; R₁ is H or halo; and R₂ is COOH. In one embodiment the hormone adapted tumor is an estrogen responsive cancer, and in one embodiment the cancer is breast cancer.

In another embodiment of the present invention the concepts regarding adaptive hypersensitivity described herein can also be applied to develop an innovative approach to the treatment of hormone-dependent breast cancer. This would involve cyclic therapy with the intermittent use of estrogen-deprivation therapy with aromatase inhibitors, followed by administration of high-dose estrogen. The rationale for this approach is that treatment with aromatase inhibitors would cause cells to up-regulate pathways involving MAPK and PI3K. Present experimental data indicate that cells that have up-regulated the MAPK and P13K pathways have a more aggressive phenotype than do untreated cells. One would then specifically destroy the subset of cells that had adapted and developed hypersensitivity by inducing apoptosis with the high-dose estrogen. An ideal regimen might then use an aromatase inhibitor in combination with MAPK and PI3K inhibitors initially. At appropriate times, one could then administer a pulse of high-dose estrogen to induce apoptosis and kill adapted cells. Because various high-dose estradiol administration regimens have been used over a 30-year period, the relative toxicities of these agents are well known, and the means of overcoming specific problems have developed. Agents are currently available to block growth factor pathways and to administer estrogen.

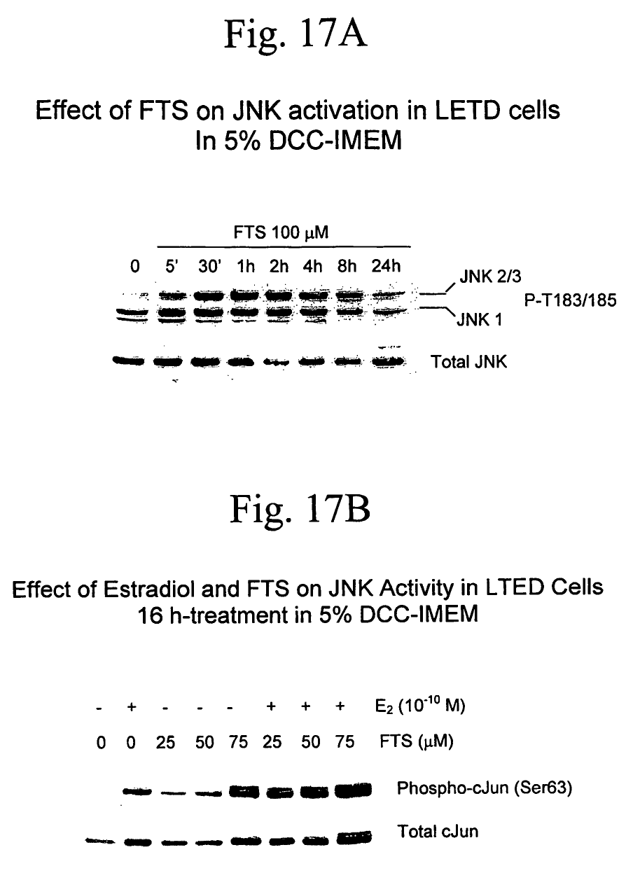

Additionally, FTS has been found to cause an increase in cell death rate (apoptosis). As shown in FIG. 17A, administration of FTS stimulates JNK activation in LTED cells cultured in vitro. Furthermore, as shown in FIG. 17B, FTS and estradiol administration to LTED cells both increase activation of JNK and result in enhances phosphorylation of cJUN. JNK activation has been reported to be associated with apoptosis. Accordingly, one aspect of the present invention is directed to the use of an mTOR inhibitor to induce apoptosis in hormone responsive cancer cells. The method of enhancing the rate of apoptosis in hormone responsive cancer cells comprises administering a compound of the general formula:

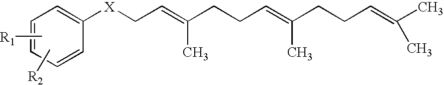

wherein X is NH or S; R₁ is H or halo; and R₂ is COOH. In one embodiment the composition is administered to an estrogen responsive cancer cell and in one embodiment the cancer is breast cancer. In another embodiment the composition is administered to a patient that has been receiving hormone deprivation therapy to enhance apoptosis in the remaining tumor cells.

### EXAMPLE 1

The Mechanics of Hormone Deprivation Adaptation

Clinical observations from two decades ago provided clues regarding the process of adaptation of breast cancer cells to hormonal therapies. These involved the observation that patients initially responding to an anti-estrogen and then relapsing, later experienced further tumor regression upon exposure to an inhibitor of estrogen biosynthesis (aromatase inhibitor). One would have expected complete cross resistance between aromatase inhibitors and tamoxifen because both provide a means to block the cellular effects of estrogen. However, this was not the case. Specifically, 50% of women responding initially to tamoxifen but then relapsing, experienced secondary clinical benefit when crossed over to aminoglutethimide, a first generation aromatase inhibitor. This observation provided evidence that aminoglutethimide would be efficacious as second line therapy for women with hormone dependent breast cancer and that different mechanisms of action must be operative with respect to aromatase inhibitors and tamoxifen.

To explain the lack of complete cross resistance between aromatase inhibitors and antiestrogens, investigators postulated the phenomenon of “adaptive hypersensitivity”. This hypothesis suggested that tamoxifen might exert pressure causing breast cancer cells to adapt and develop hypersensitivity to the estrogenic properties of tamoxifen. It is well known that tamoxifen, as a representative of the class of drugs called selective estrogen receptor modulators, or SERMs, exerts both estrogen agonistic or antagonistic properties depending upon the tissue examined and circumstances involved. To circumvent the hypersensitivity to tamoxifen, one could stop this agent and use potent aromatase inhibitors to reduce estradiol to very low levels. With this as a rationale, one would then expect secondary tumor regressions when using second line aromatase inhibitors.

Clinical observations in pre-menopausal women with breast cancer also supported the possibility that breast cancer cells can adapt to conditions of treatment by developing enhanced sensitivity to estradiol. Hormone dependent breast cancers often regress in response to surgical removal of the ovaries, a treatment that lowers circulating plasma estradiol from approximately 200 pg/mL to 10-15 pg/m. In response to this acute deprivation of estradiol, tumors regress for 12 to 18 months on average before they begin to regrow. Second line therapy with surgical oophorectomy or with aromatase inhibitors may then induce additional tumor regressions by lowering estradiol concentrations further to 1-5 pg/ml. These observations first demonstrated enhanced sensitivity to circulating estradiol. Specifically, 200 pg/ml of estradiol were required to stimulate tumor growth before oophorectomy whereas levels of 10-15 pg/ml were sufficient to cause tumor proliferation after adaptation 12-18 months later.

To directly demonstrate the phenomenon of adaptive hypersensitivity and determine the mechanisms involved a model system involving human breast cancer MCF-7 cells in vitro has been utilized. Wild type MCF-7 cells are cultured over a prolonged period in estrogen-free medium to mimic the effects of primary endocrine therapy. This process involves Long Term Estradiol Deprivation and the adapted cells are called by the acronym, LTED cells. In response to estradiol deprivation, MCF-7 cells initially stop growing but then, three to six months later, adapt and grow as rapidly as wild type MCF-7 cells maximally stimulated with estradiol. This effect has been attributed to the development of hypersensitivity to estradiol with re-growth in response to residual amounts of estrogen in the charcoal stripped culture media.

Direct evidence of hypersensitivity is demonstrated by the showing that four log lower concentrations of estradiol can stimulate proliferation of LTED cells compared to wild type MCF-7 cells. In vivo studies also demonstrated that LTED cell xenografts grown in nude mice are hypersensitive to low doses of estradiol. Applicants have recently demonstrated that long term exposure to tamoxifen in castrated nude mice also induces a state of hypersensitivity to estradiol. During the phase when tamoxifen first begins to stimulate tumor growth, removal of tamoxifen and administration of very low doses of estradiol stimulates tumor growth. In marked contrast, wild type tumors not exposed to tamoxifen long term are not stimulated by such low doses of estradiol. Taken together, these experiments indicate that either estradiol deprivation or blockade of estrogen action with antiestrogens enhances the level of sensitivity to estrogens or to the estrogenic properties of tamoxifen. This phenomenon of adaptive hypersensitivity is believed to be responsible for the superiority of aromatase inhibitors over tamoxifen in a variety of clinical settings.

The process of hormone adaptation could involve modulation of the genomic effects of estradiol acting on transcription, non-genomic actions involving plasma membrane related receptors, cross talk between growth factor and steroid hormone stimulated pathways, or interactions among these various effects. One possibility is that enhanced receptor mediated transcription of genes related to cell proliferation might be involved. Indeed, the levels of ER alpha increased 4-10 fold during long term estradiol deprivation. Accordingly, to directly examine whether enhanced sensitivity to E₂ in LTED cells occurred at the level of ER mediated transcription, the effects of estradiol on transcription in LTED and in wild type MCF-7 cells was quantitated. The effect of E₂ on c-myc message levels, progesterone receptor (PgR) and pS2 concentrations, and on ERE-CAT reporter activity was measured. Although basal levels of pS2 and PgR were increased, no shift to the left in estradiol dose response curves (the end point utilized to detect hypersensitivity) was observed for any of these responses when comparing LTED with wild type MCF-7 cells. These data indicate that hypersensitivity of LTED cells to estradiol does not occur at the level of ER-mediated gene transcription.

Another explanation relates to the possibility that adaptation might involve dynamic interactions between pathways utilizing steroid hormones and growth factors for signaling. Both estradiol and various peptide growth factors are mitogens for breast tissues. A variety of studies indicate that growth factor secretion and action can be stimulated by estradiol. These effects are believed to result from the genomic effects of estradiol to stimulate transcription of early response genes such as c-Myc and growth factors such as TGF alpha. Growth factors result in the activation of MAP kinase which directly and indirectly enhance the degree of phosphorylation of the estrogen receptor. MAP kinase directly phosphorylates serine 118 and also stimulates Elk and RSK activity which phosphorylates serine 167.

To determine if basal levels of MAP kinase were elevated in LTED cells, the level of activated MAP kinase was measured in LTED cells in vitro and in LTED xenografts in nude mice. Furthermore, activated MAP kinase was demonstrated to have a role in the enhanced growth of LTED cells, since inhibitors of MAP kinase such as PD98059 or U 0126 block the incorporation of treated thymidine into DNA. Upstream inhibitors of the MAP kinase pathway such as mevastatin or genestein, also block tritiated thymidine uptake. These data suggest that an increase in activated MAP kinase participates in the adaptive hypersensitivity process. To demonstrate proof of this principle, activation of MAP kinase in wild type MCF-7 cells was stimulated by administering TGF alpha.

Initial characterization data demonstrated increases in MAP kinase in MCF-7 cells with doses of TGF alpha ranging from 0.1 to 10 ng/ml and blockade of this effect with the MAP kinase inhibitor PD 98059. Administration of TGF alpha at a dose of 10 ng/ml caused a two log shift to the left in the ability of estradiol to stimulate the growth of wild type MCF-7 cells. To demonstrate that this effect related specifically to MAP kinase and not to a non-specific effect of TGF alpha, PD 98059 was co-administered. Under these circumstances, the 2 log left shift in estradiol dose response, returned back to baseline. As further evidence of the role of MAP kinase, PD 98059 was administered to LTED cells and its effect on level of sensitivity to estradiol was examined. This agent partially shifted dose response curves to the right by approximately one-half log. Taken together, these data suggest that MAP kinase activation does participate mechanistically in the adaptive hypersensitivity process.

While an important component, MAP kinase does not appear to be solely responsible for hypersensitivity to estradiol. Blockade of this enzyme did not completely abrogate hypersensitivity. Accordingly, the PI-3 kinase pathway was examined to determine if it was upregulated in LTED cells as well. Experiments determined that LTED cells exhibit an enhanced activation of AKT, P70 S6 kinase, and 4EBP-1 (all components of the PI-3 kinase pathway). Dual inhibition of PI-3-kinase with Ly 294002 and MAP kinase with U -0126 shifted the level of sensitivity to estradiol more dramatically, by more than two logs to the right.

Upregulation of MAP kinase and PI-3-kinase could reflect either a constitutive activation of growth factor receptors, an increase in the endogenous secretion of growth factors, or other mechanisms. Inhibition of MAP kinase with a pure antiestrogen would rule out the possibility of constitutive activation of growth factor receptors or growth factor secretion. Accordingly, the pure anti-estrogen, faslodex (fulvestrant) was administered and the level of activation of MAP kinase in LTED cells was examined. Surprisingly, fulvestrant returned the level of activated MAP kinase back to the level seen in wild type MCF-7 cells. This observation ruled out constitutive growth factor effects in LTED cells and suggested an interaction between estrogen receptor mediated functions and the elevation of MAP kinase.

One possible mechanism to explain the activation of MAP kinase would be through non-genomic effects of the ER acting at the level of the cell membrane. Non-genomic actions of E₂ have only been recognized recently and encompass activation of mitogen-activated protein kinase (MAP kinase), Ras, Raf-1, PKC, PKA, Maxi-K channels, elevation of intracellular calcium levels, and release of nitric oxide. The adaptor protein Shc represents a key modulator of tyrosine kinase activated peptide hormone receptors. As an upstream regulator of MAPK, Shc transduces mitogenic and differentiation signals from a variety of tyrosine kinase receptors such as EGFR, NGFR, PDGFR and IGFR, to downstream kinase cascades. Upon receptor activation and auto-phosphorylation, Shc binds rapidly to specific phosphotyrosine residues of receptors through its PTB or SH2 domain and becomes phosphorylated itself on tyrosine residues of the CH domain. The phosphorylated tyrosine residues on the CH domain provide the docking sites for the binding of the SH2 domain of Grb2 and hence recruit Sos, a guanine nucleotide exchange protein. Formation of this adapter complex allows Ras activation via Sos, leading to the activation of the MAPK pathway.

Estrogen deprivation might trigger activation of a non-genomic, estrogen regulated, MAP kinase pathway which utilizes Shc. To investigate this possibility, MAP kinase activation was used as an endpoint with which to demonstrate rapid non-genomic effects of estradiol. The addition of E₂ stimulated MAP kinase phosphorylation in LTED cells within minutes. The increased MAP kinase phosphorylation by E₂ was time and dose-dependent being greatly stimulated at 15 min and remaining elevated for at least 30 min. Maximal stimulation of MAP kinase phosphorylation was at 10⁻¹⁰ M of E₂.

Shc proteins are known to couple tyrosine kinase receptors to the MAPK pathway and activation of Shc involves the phosphorylation of SHC itself. To investigate if the Shc pathway was involved in the rapid action of estradiol in LTED cells, tyrosine phosphorylated proteins were immuno-precipitated and tested for the presence of Shc under E₂ treatment. E₂ rapidly stimulated Shc tyrosine phosphorylation in a dose and time dependent fashion with a peak at 3 minutes. The pure estrogen receptor antagonist, fulvestrant, blocked E₂-induced Shc and MAPK phosphorylation at 3 min and 15 min respectively. The time frame suggests that Shc is an upstream component in E₂-induced MAPK activation.

To provide direct evidence of the necessity of Shc for MAP kinase activation, a GST-tagged full-length Shc mutant (ShcFFF) with point mutations at tyrosines 239/240 and 317 (Y239/240/317F) was transfected into LTED cells. These three sites of tyrosine phosphorylation of Shc are important for its interaction with Grb2 and for transduction of the signal to down-stream components. Expression of dominant ShcFFF markedly inhibited estradiol stimulation level of MAPK phosphorylation. Thus Shc is necessary for activation of MAP kinase.

The adapter protein Shc, may directly or indirectly associate with ERα in LTED cells and thereby mediate E₂-induced activation of MAP kinase. To test this hypothesis, Shc was immunoprecipitated from non-stimulated and E₂-stimulated LTED cells and then immunoblots were probed with anti-ERα antibodies. The resulting data showed that the ERα/Shc complex pre-existed before E₂ treatment and E₂ time-dependently increased this association. In parallel with Shc phosphorylation, a maximally induced association was observed between ERα and Shc at 3 min. MAP kinase pathway activation by Shc requires Shc association with adapter protein Grb2 and then further association with Sos. By immunoprecipitation of Grb2 and detection of both Shc and Sos, the Shc-Grb2-Sos complex was demonstrated to be constitutively existing at relatively low levels in LTED cells, but greatly increased by treatment of cells with 10⁻¹⁰ M E₂ for 3 min.

Because ERα, Shc and MAP kinase are all involved in E₂ action in MCF-7 cells, upstream components responsible for Shc phosphorylation were determined by measuring the effects of PP2, ICI and PD98059 on E₂-induced phosphorylation of Shc. In the presence of the inhibitors, MCF-7 cells were stimulated with vehicle or 10⁻¹⁰ M E₂ for 3 minutes and the status of Shc phosphorylation was examined. Both PP2 and ICI effectively inhibited E₂-induced Shc phosphorylation, implying that Src family kinases and ERα are required for Shc activation. As expected, PD98059 did not influence the phosphorylation status of Shc, suggesting that it functions downstream of Shc. No effects of these inhibitors were apparent in the absence of E₂ stimulation. Taken together, these results indicate that both ERα and Src are upstream components of She functionality and their involvement is required for Shc phosphorylation. Each of these inhibitors was capable of reducing the rate of cell proliferation in LTED cells.

To provide evidence that the ERα-Shc-MAP kinase pathway exerts biologic effects, the role of MAP kinase on the activation of Elk-1 (a transcriptional factor phosphorylated and activated by MAPK) was evaluated. When activated, Elk-1 serves as a down stream mediator of cell proliferation. The phosphorylation of Elk1 by MAPK can up-regulate its transcriptional activity. By co-transfection of LTED cells with both GAL4-Elk and its reporter gene GAL4-luc, it was demonstrated that E₂ dose-dependently increased Elk -1 activation at 6 hours as shown by luciferase assay.

It has been reported that cell mobility is controlled by a network of membrane initiated signals, such as activation of the Shc-Ras-MAPK pathway. Recently, it was reported that cell focal adhesions are highly dynamic structures. Cells can rapidly respond to the stimulation by growth factors and show reorganization of their cytoskeleton and cell shape. To examine E₂ effects on reorganization of the actin cytoskeleton, the distribution of F-actin and also redistribution of the ERα localization in LTED and MCF-7 cells was determined by phalloidin staining. Untreated LTED cells expressed low actin polymerization and a few focal adhesion points. After E₂ stimulation in contrast, the cytoskeleton underwent remodeling associated with formation of cellular ruffles, lamellipodias and leading edges, alterations of cell shape and loss of mature focal adhesion points. A subcellular redistribution of ERα to these dynamic membranes upon E₂ stimulation represented another important feature of LTED cells. The ER antagonist ICI 182 780 at 10⁻⁹ M blocked E₂-induced ruffle formation as well as redistribution of ERα to the membrane with little effect by itself. Therefore, these studies further demonstrated the rapid action of E2 with respect to dynamic membrane alterations in LTED cells.

To provide further proof of non-genomic ER mediated effects, a series of designer estrogen receptors was constructed. The ER generated by the group of Dr. Pierre Chambon, lacking a nuclear localization signal was used as a starting point. To this, a membrane localization signal was coupled, comprising a 43 amino-acid sequence, which is used in the CNS to bring proteins to the membrane. COS cells lacking an ER were then transfected with the three ER constructs. Using dual fluorescence microscopy, nearly exclusive localization of the wild type ER to the nucleus and of the ER lacking the nuclear localization signal to the cytoplasm was observed. Receptor containing the membrane localization signal concentrated in the plasma membrane but also was found in the cytoplasm. Only the membrane ER responded to exogenous estradiol with MAP kinase activation. In addition, only the membrane localized ER stimulated cell proliferation as evidenced by BRD-U incorporation and total cell counts. These data further support the function of the membrane ER to enhance cell proliferation.

Summary of Mechanisms for Adaptive Hypersensitivity

Long term estradiol deprivation causes an upregulation of the amount of ER alpha present and of processes involved in utilization of membrane related ER.

This results in an increased level of activation of the MAP kinase as well as the PI-3-kinase pathways. All of these signals converge on downstream pathways directly involved in cell cycle functionality and probably exert synergistic effects at that level. As a reflection of this, E2F1, an integrator of cell cycle stimulatory and inhibitory events, is hypersensitive to the effects of estradiol in LTED cells. Thus, the data supports the hypothesis that hypersensitivity reflects a downstream synergistic interaction of several pathways converging at the level of the cell cycle. An increase in the basal level of transcription of ER regulated genes may also be involved in the process but does not represent the proximate cause of hypersensitivity since transcriptional events respond to estradiol with similar dose response curves in wild type and LTED cells.

### EXAMPLE 2

Blockade of MAP and PI3 Kinase Pathways Markedly Reduces the Sensitivity of Cells to the Effects of Estradiol on Cell Proliferation

To investigate whether the MAP and P13 kinase pathways have a role in estradiol induced cell proliferation, the following experiments were conducted. Initial characterization data demonstrated that administration of TGF increased MAPK activation with increases in MCF-7 cells resulting from doses of TGF ranging from 0.1 to 10 ng/ml. Blockade of this effect was obtained with the MAPK inhibitor PD98059. Administration of TGF at a dose of 10 ng/ml caused a 2-log shift to the left in the ability of estradiol to stimulate the growth of wild-type MCF-7 cells. To demonstrate that this effect related specifically to MAPK and not to a nonspecific effect of TGF, PD98059 was co-administered. Under these circumstances, the 2-log left shift in estradiol dose response returned to baseline. Although an important component, MAPK did not seem to be solely responsible for hypersensitivity to estradiol. Blockade of this enzyme did not completely abrogate hypersensitivity. Accordingly, the phosphatidylinositol-3-OH kinase (PI3K) pathway was examined to determine whether it, too, was up-regulated in Long Term Estradiol Deprivation (LTED) cells. In experiments, LTED cells exhibited an enhanced activation of Akt, P70 S6 kinase, and 4EBP-1 (all components of the PI3K pathway). Dual inhibition of PI3K with LY40029 and MAPK with U0126 shifted the level of sensitivity to estradiol more dramatically, by more than 2 logs to the right. On the basis of these observations, it is believed that adaptive hypersensitivity involves the joint activation of the PI3K and MAPK pathways.

### EXAMPLE 3

Estradiol Effects on Wild Type and LTED Cells

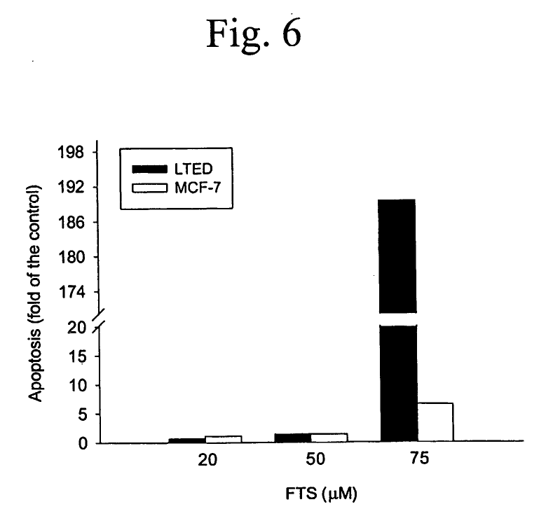

The LTED in vitro model of long-term estradiol deprivation demonstrates a hypersensitivity of LTED cells to estradiol. Applicants postulated that these cells might also have become sensitized to the pro-apoptotic effects of estradiol. To test this hypothesis, an ELISA assay was used to assess apoptosis and to compare the effects of estradiol in wild-type and LTED cells. The expected inhibition of apoptosis occurred in the wild-type cells but, in marked contrast, a dramatic enhancement of this parameter occurred in LTED cells (see FIG. 6). The presence of apoptosis in LTED cells was confirmed by the measurement of annexin V and the use of time-lapse microscopy.

As a mechanistic explanation for the differential effects of estradiol in LTED and wild-type cells, investigations revealed that the death receptor Fas was up-regulated in the LTED cells. Because Fas ligand has an estrogen response element in its promoter region, estradiol stimulated the level of Fas ligand in both LTED and wild-type MCF-7 cells. Only the LTED cells contained a Fas receptor and could respond with apoptosis. As additional evidence regarding the role of the Fas receptor, an activating antibody, directed against Fas, stimulated apoptosis in LTED cells.

FIGS. 17A & 17B represent data showing the effects of FTS on JNK activation in LTED cells cultured in vitro. As shown in FIG. 17A, administration of FTS stimulates JNK activation in LETD cells cultured in vitro. Furthermore, as shown in FIG. 17B, FTS and estradiol administration to LTED cells both increase activation of JNK and result in enhances phosphorylation of cJUN. JNK activation has been reported to be associated with apoptosis.

Women with breast cancer receive third-generation aromatase inhibitors over a period of 1 to 5 or more years. On the basis of the LTED model system, it follows that the breast cancer cells in these patients become sensitized to the proapoptotic effects of estradiol. Under these circumstances, estradiol might stimulate apoptosis and cause tumor regression. From the 1940s until the early 1980s, high-dose estrogen in the form of diethylstilbestrol (DES) was the treatment of choice for postmenopausal women with breast cancer. Clinical studies demonstrated that pre- and perimenopausal women rarely respond to this therapy, whereas responses increased as a function of the number of years after the onset of menopause. The long period of time after menopause, in fact, may mimic the effects of long-term estradiol deprivation. Accordingly, this might then explain the responses to high-dose estrogen by its ability to induce apoptosis. Therefore one would expect that women receiving aromatase inhibitors long-term would also respond to high-dose estrogen with apoptosis.

### EXAMPLE 4

FTS Blocks mTOR Activity

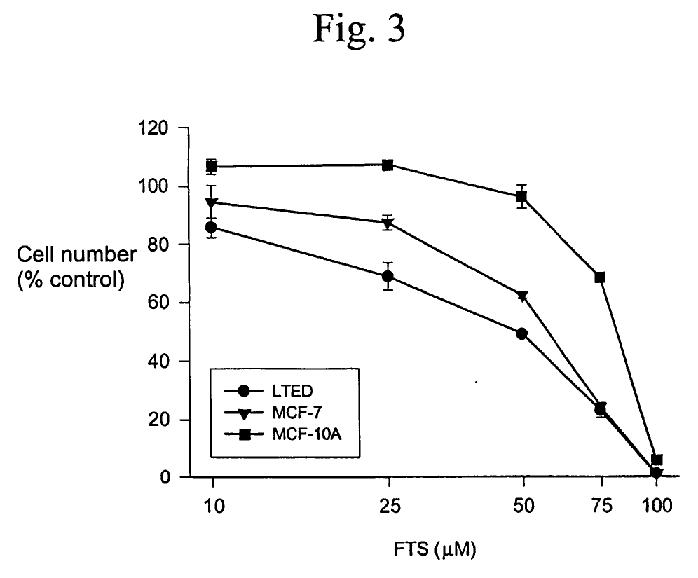

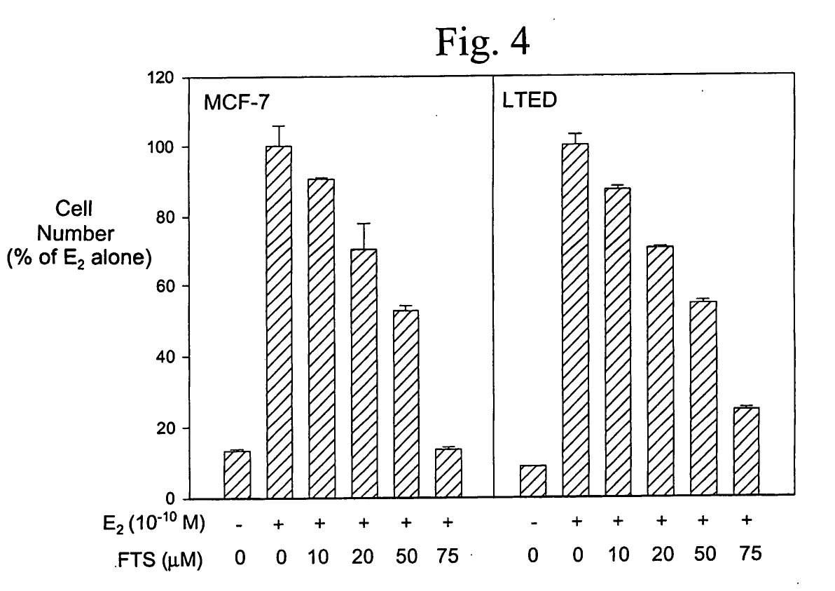

The effect of FTS on growth of breast cancer cells was examined in two different cell lines: MCF-7 wild type cells which represent a model for untreated breast cancer and LTED cells as a model of cells adapting to endocrine treatment. Cells were grown in their culture media and treated with FTS for five days. As shown in FIG. 3, FTS dose-dependently inhibited basal growth of both cell lines. The effect of FTS on E₂-stimulated growth of MCF-7 and LTED cells was the examined. Five-day treatment of MCF-7 cells with E₂ (10⁻¹⁰ M) increased cell number by 8-fold compared with the vehicle control. Addition of FTS reduced cell number in a dose-dependent manner with about 50% inhibition at the concentration of 50 μM (FIG. 3). FIG. 4 shows growth stimulation of LTED cells by E₂ (10⁻¹⁰ M) in the presence of fulvestrant (10⁻⁹ M), which was inhibited by FTS. The extent of inhibition of E₂-stimulated cell growth by FTS was similar in these two cell lines.

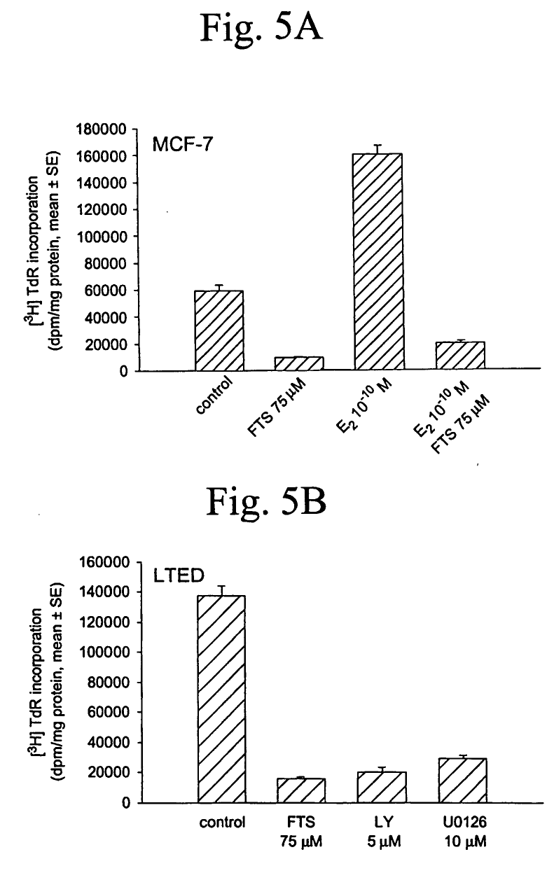

The inhibitory effect of FTS on cell growth may be the result of inhibition of cellular proliferation, induction of apoptosis, or both. To determine the mechanism by which FTS inhibits cell growth, FTS's effect on DNA synthesis and apoptosis was investigated. In both MCF-7 and LTED cells, FTS (75 μM) significantly reduced the amount of [³H] thymidine incorporated into DNA (FIGS. 5A & 5B). Estradiol (10⁻¹⁰ M) increased [³H] thymidine incorporation by 2.6 fold compared to the control level in MCF-7 cells. This increase was completely blocked by FTS (FIG. 5A).

The effect of a three day treatment with FTS on apoptosis in MCF-7 and LTED cells was examined using the ELISA assay for quantitation. Low concentrations of FTS (20 and 50 μM) did not induce apoptosis in either cell line. A dramatic increase in apoptosis occurred at a concentration of 75 μM (FIG. 6). LTED cells were much more sensitive than MCF-7 cells to the apoptotic effects of FTS. These data indicate that both inhibition of DNA synthesis and induction of apoptosis by FTS attribute to growth inhibition of MCF-7 and LTED cells.

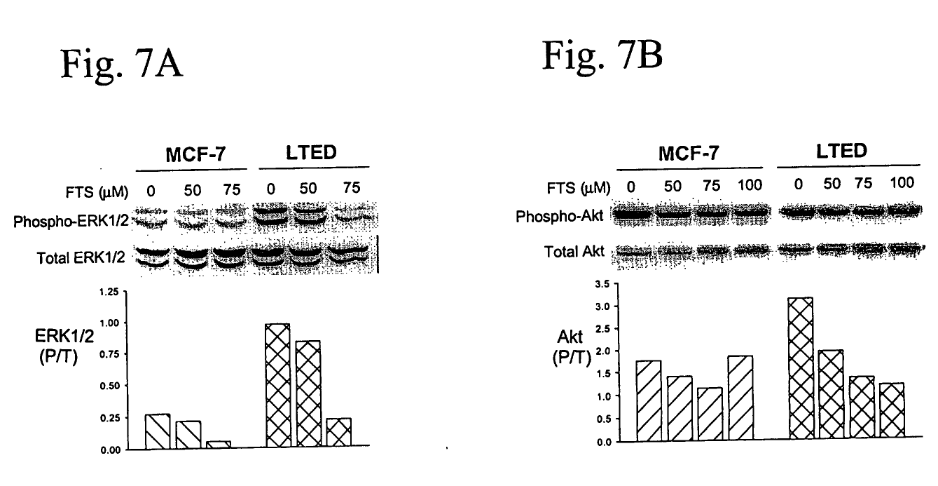

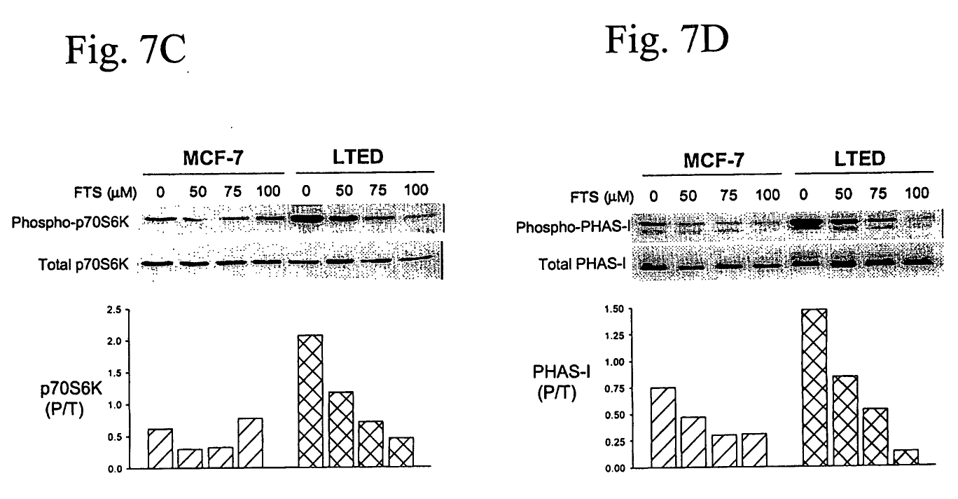

The levels of phosphorylated ERK1/2 MAPK, Akt (Ser⁴⁷³), p70S6K (Thr³⁸⁹) and PHAS-1 (Ser⁶⁵) were consistently elevated in LTED cells compared to those in wild type MCF-7 cells as indicated by Western blot analysis using specific antibodies against phosphorylated ERK 1/2 (FIG. 7A), Akt (FIG. 7B), p70S6 kinase (FIG. 7C) and PHAS-1 (FIG. 7D). Treatment with FTS for 24 h dose-dependently inhibited phosphorylation of all four proteins in LTED cells. In contrast, ERK1/2 and 4E BP 1 fell to a lesser extent (approximately 50%) in MCF-7 cells and p70 S 6 kinase and phospho-AKT exhibited inconsistent decrements. (FIGS. 7C & 7D).

The effects of the specific growth factors, EFG and IGF-1 were also examined. Serum contains multiple growth factors that activate MAP kinase and PI3 kinase either by recruitment and activation of the common mediator Ras (such as EGF) or through a direct mechanism (such as IGF-1). To further explore the site of action of FTS, EGF-induced activation of MAPK and PI3 kinase was first examined. Specific inhibitors for PI3 kinase and mammalian target of rapamycin (mTOR) were included for comparison. The cells were serum starved for 24 hours, pretreated for one hour with FTS and 3 hours with LY 294002 or rapamycin before challenge with EGF (1 μg/ml). Time to the peak stimulation by EGF on MAPK, Akt, p70S6K and PHAS-1 varied but they all lasted at least 90 minutes. One-hour treatment was chosen as the time point allowing most efficient comparisons among molecules. Responses to EGF and inhibitors were similar in MCF-7 and LTED cells.

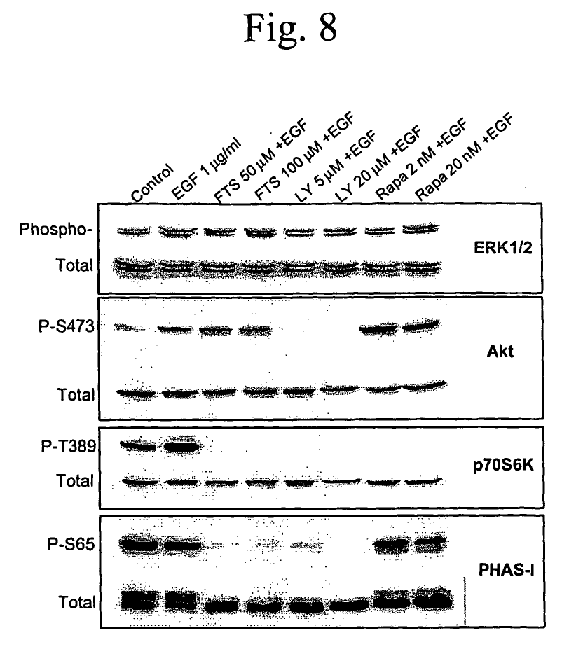

Peak stimulation of ERK1/2 MAP kinase phosphorylation was at 5-10 minutes of EGF treatment. By 1 h, the level of phosphorylated ERK1/2 was still higher than the control. Pretreatment with FTS, LY 294002 and rapamycin did not inhibit activated ERK at either concentration (FIG. 8). Phosphorylation of Akt at Ser⁴⁷³ was enhanced by EGF and completely inhibited by the specific PI3 kinase inhibitor, LY 294002. In marked contrast, neither FTS nor rapamycin blocked this step. EGF induced an even more dramatic increase in phosphorylation of p70S6K at Thr³⁸⁹. Thr³⁸⁹ phosphorylation is central to p70S6K activity and is regulated by mitogenic stimuli through both the PI3 kinase and mTOR pathways. The data showed that pretreatment with LY 294002, rapamycin, and FTS, even at 50 μM completely abolished Thr³⁸⁹ phosphorylation of p70S6K (FIG. 8). Only FTS blocked phosphorylation of p70S6K and PHAS-1 without impacting Akt activation.

PHAS-1 was highly phosphorylated at Ser⁶⁵ in LTED cells even after 24 hours of serum starvation and there was no further stimulation by EGF. FTS and LY dose-dependently inhibited Ser⁶⁵ phosphorylation. Rapamycin only caused partial inhibition even at higher concentration. It should be noted that there are at least four serine and threonine residuals on PHAS-1 and these modifications retard migration during electrophoresis. Accordingly, PHAS-1 bands moved faster when phosphorylation was reduced through the action of the inhibitors.

IGF-1 is another growth factor that activates both the MAP kinase and the PI3 kinase pathways. However, the mechanism by which IGF-I stimulates these two pathways is different from that by EGF. IGF-1 and EGF exhibited similarly stimulatory effect on phosphorylation of ERK1/2 MAP kinase, Akt, p70S6K, and PHAS-1 except that IGF-1 induced more potent and sustained stimulation than EGF on Akt and p70S6K phosphorylation.

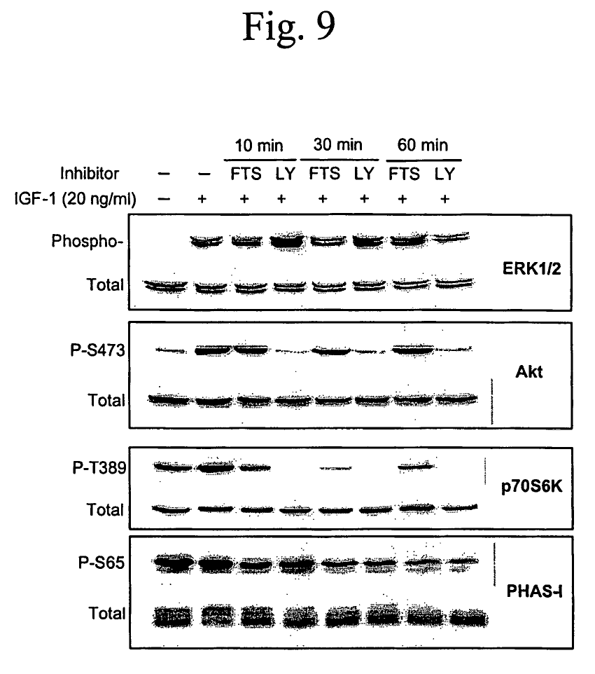

LTED cells were pretreated with FTS (100 μM) or LY 294002 (20 μM) for 10, 30, and 60 min and then IGF-1 (20 ng/ml) was added for 10 min. IGF-1 treatment significantly induced phosphorylation of MAP kinase, Akt, and p70S6K but not of PHAS-1. Neither FTS nor LY 294002 inhibited ERK1/2 phosphorylation. LY caused profound inhibition on IGF-1 induced phosphorylation of Akt at Ser⁴⁷³ and p70S6K at Thr³⁸⁹. The inhibitory effect was seen as early as 10 minutes of treatment whereas FTS induced no effect. In marked contrast, both FTS and Ly inhibited the phosphorylation of 4E BP-1, particularly at the 60 minute time point. (FIG. 9).

FTS displayed very potent inhibition on phosphorylation of p70S6K and PHAS-1. Unlike LY 294002, FTS showed limited inhibition on Akt phosphorylation indicating that the site of action of FTS is down stream of Akt. p70 S6K undergoes sequential phosphorylation on at least eight serine/threonine residues upon mitogenic stimuli. Tbr³⁸⁹ and Thr²²⁹ are two critical phosphorylation sites to the kinase activity. Studies have shown that PDK1 directly phosphorylates Th³²⁹ whereas mTOR might be the upstream kinase that phosphorylates Thr³⁸⁹. To further explore the point of action of FTS on p70 S6K, its effect on Thr²²⁹ phosphorylation was examined with a specific antibody.

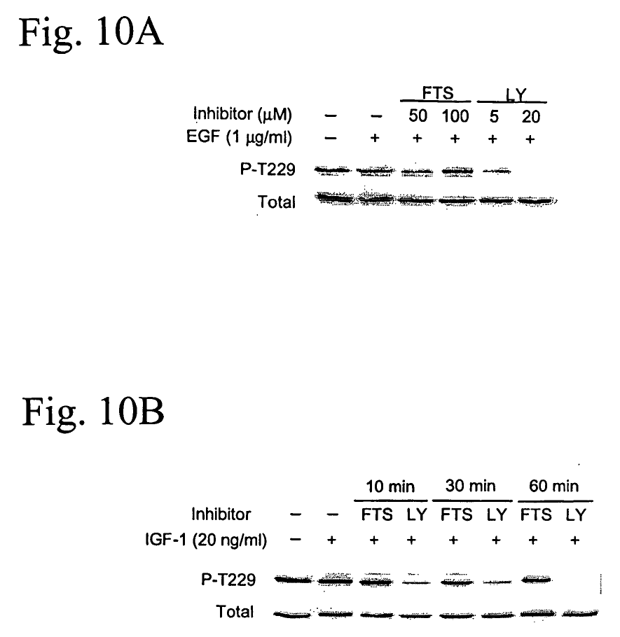

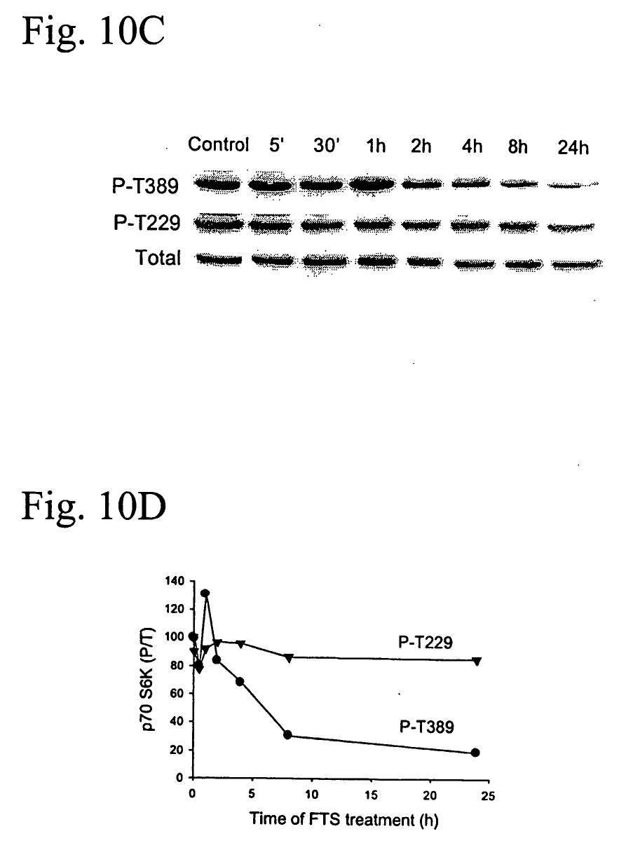

In comparison with Thr³⁸⁹ phosphorylation, FTS caused very limited, if any, inhibition on Thr²²⁹ phosphorylation in LTED cells treated with EGF (FIG. 10A) or IGF-1 (FIG. 10B). In contrast, LY294002 blocked growth factor induced Thr²²⁹ phosphorylation in a dose- and time-dependent manner (FIGS. 10A and 10B). FIG. 10C shows that the inhibitory effect of FTS (100 μM) on Thr³⁸⁹ phosphorylation in serum containing medium occurred at 2 h and maximized at 24 h. Thr²²⁹ phosphorylation, in contrast, was only slightly reduced. These data provide additional evidence that inhibition of FTS on p70 S6K occurs downstream of PI3 kinase. Therefore, FTS's effect on MTOR activity was investigated.

An assay specific for mTOR was used to monitor the effects of FTS on mTOR. The assay utilizes the transfection of recombinant mTOR into cells, its immunoprecipitation with an anti-mTOR antibody, and then a kinase assay to assess the activity of the precipitated mTOR. In this assay, FTS completely blocked the ability of mTOR to phosphorylate a substrate specific to mTOR. This effect was maximal at a concentration of 100 micromolar and could be seen at doses of 25 micromolar. Careful attention to the means of diluting FTS for these experiments was critical since FTS has only limited solubility in buffer.

### EXAMPLE 5

Farnesylthiosalicylic Acid Inhibits mTOR Activity Both in Cells and In Vitro by Promoting Dissociation of the mTOR-Raptor Complex

The mammalian target of rapamycin, mTOR, is a Ser/Thr protein kinase involved in the control of cell growth and proliferation. One of the best characterized substrates of mTOR is PHAS-I (a.k.a. 4E-BP1). PHAS-I binds to eIF4E and represses cap-dependent translation by preventing eIF4E from binding to eIF4G. When phosphorylated by mTOR, PHAS-I dissociates from eIF4E, allowing eIF4E to engage eIF4G, thus increasing the formation of the eIF4F complex needed for the proper positioning of the 40S ribosomal subunit and for efficient scanning of the 5′-UTR. In cells mTOR is found in mTORC1, a complex also containing raptor and mLST8. Raptor (a.k.a. mKOG1) is a newly discovered Mr=150,000 protein, which possesses a unique NH₂ terminal region followed by three HEAT motifs and seven WD-40 domains. mLST8 (a.k.a. GP1) is homologous to members of the family of β subunits of heterotrimeric G proteins and it consists almost entirely of 7 WD-40 repeats. The roles of the two mTOR-associated proteins are still not fully defined, but both appear necessary for optimal mTOR function, since depleting cells of either raptor or mLST8 with siRNA decreases mTOR activity. Raptor binds directly to PHAS-I and mutations in PHAS-I that decrease raptor binding also inhibit phosphorylation of PHAS-I by mTOR in vitro. It has been proposed that raptor functions in TORC1 as a substrate-binding subunit which presents PHAS-I to mTOR for phosphorylation.

Rapamycin is the prototypic inhibitor of mTOR function. Determining the sensitivity to rapamycin has been an invaluable approach for identifying processes in cells controlled by mTOR. In addition to its experimental use, rapamycin and/or the related drug, CCI-779, are used clinically to inhibit host rejection of transplanted organs, the occlusion of coronary arteries following angioplasty, and the growth of tumor cells. Rapamycin action is complicated in that in order to bind mTOR with high affinity, the drug must first form a complex with the prolyl isomerase, FKBP12. Rapamycin-FKBP12 binds upstream of the kinase domain in a region of mTOR referred to as the FRB. Binding of the complex markedly attenuates, but does not fully inhibit, mTOR activity in vitro. The incomplete inhibition raises the possibility that there are rapamycin-insensitive functions of mTOR in cells. Thus, agents that interfere with mTOR by mechanisms different from that of rapamycin may prove to be useful experimental and/or clinical tools.

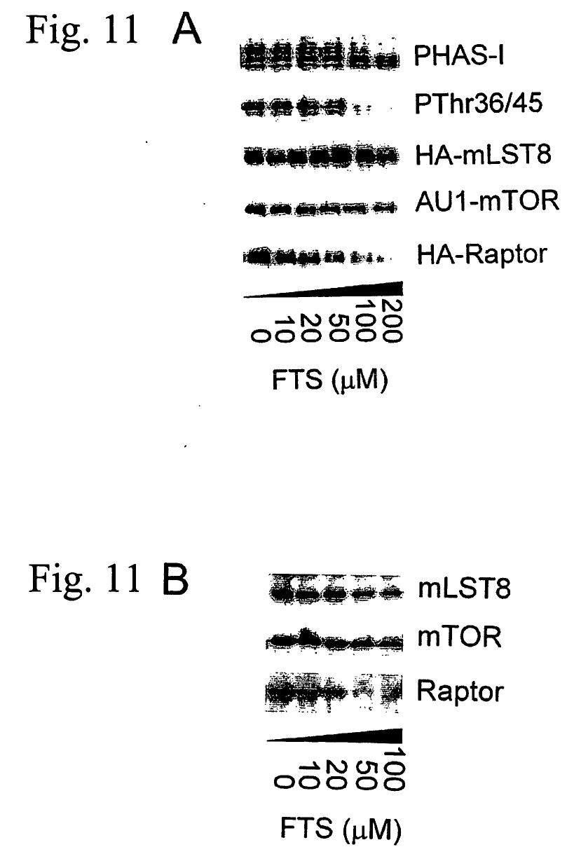

To investigate the effects of FTS on mTOR function in 293T cells, changes in the phosphorylation of PHAS-I, a well characterized target of mTOR were monitored. Phosphorylation of Ser64 and Thr69 in PHAS-I causes a dramatic decrease in the mobility of the protein in SDS-PAGE, so that changes in the mobility provide an index of changes in phosphorylation state. Incubating cells with increasing concentrations of FTS decreased the phosphorylation of PHAS-I, as evidenced by a decrease in the electrophoretic mobility (FIG. 11A). To determine whether FTS also promoted dephosphorylation of Thr36 and Thr45, the preferred sites for phosphorylation by mTOR, an immunoblot was prepared with PThr36/45 antibodies (FIG. 11A). Increasing FTS markedly decreased the reactivity of PHAS-I with the phosphospecific antibodies. While the change in the intensity of the immunoblot does not provide an exact measure of the change in phosphorylation, as the antibodies react with PHAS-I phosphorylated in either Thr36 or Thr45, it is clear that the drug promotes the dephosphorylation of these sites.

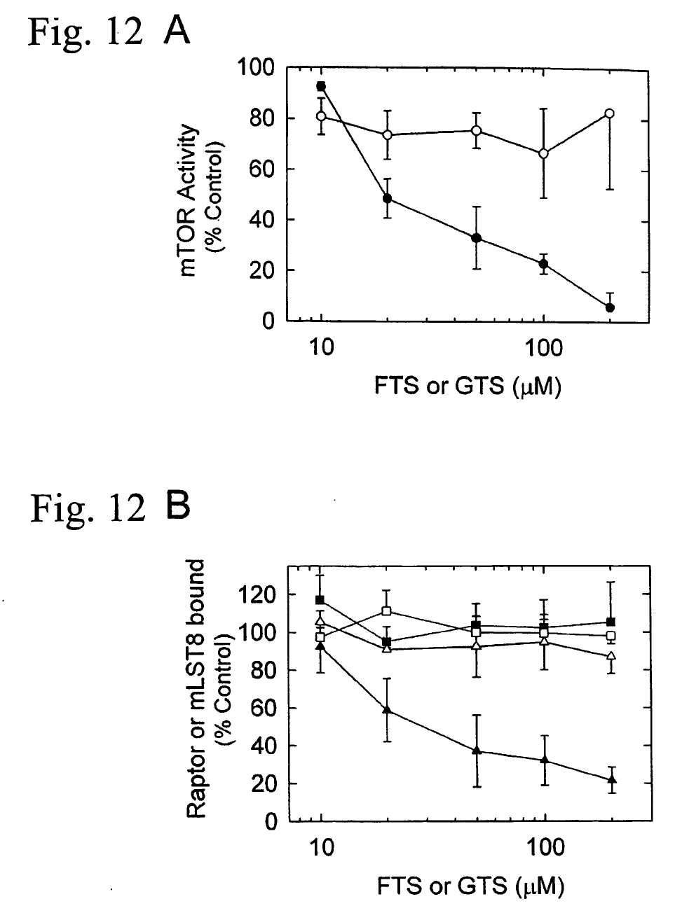

To investigate further the inhibitory effects of FTS on mTOR signaling, the effect of the drug on the association of mTOR, raptor, and mLST8 was determined. AU1-mTOR and HA-tagged forms of raptor and mLST8 were overexpressed in 293T cells, which were then incubated with increasing concentrations of FTS before AU1-mTOR was immunoprecipitated with anti-AU1 antibodies. Immunoblots were prepared with anti-HA antibodies to assess the relative amounts of HA-raptor and HA-mLST8 that coimmunoprecipitated with AU 1-mTOR (FIG. 11A). Both HA-tagged proteins were readily detectable in immune complexes from cells incubated in the absence of FTS, indicating that mTOR, raptor, and rLST8 form a complex in 293T cells. FTS did not change the amount AU1-mTOR that immunoprecipitated; however, increasing concentrations of FTS produced a progressive decrease in the amount of HA-raptor that coimmunoprecipitated (FIG. 11A). When results from three experiments were analyzed, the half maximal effect on raptor dissociation from mTOR was observed at approximately 30 μM FTS (FIG. 12B). FTS did not appear to change the amount of HA-mLST8 associated with AU1-mTOR (FIGS. 11A and 12B).

Results obtained with overexpressed proteins are not necessarily representative of responses of endogenous proteins. Therefore, experiments were conducted to investigate the effect FTS on the endogenous TORC1 in nontransfected cells. 293T cells were incubated with increasing concentrations of FTS before mTOR was immunoprecipitated with the mTOR antibody, mTAb1 (FIG. 11B). Immunoblots were then prepared with antibodies to mTOR, mLST8, and raptor. FTS markedly decreased the amount of raptor that coimmunoprecipitated with mTOR. Thus, FTS had comparable effects on the association of endogenous and overexpressed mTOR and raptor proteins. FTS also decreased the amount of mLST8 that coimmunoprecipitated with mTOR, but this effect was much less pronounced than the effect of the drug on the recovery of raptor (FIGS. 11A, 11B, and 12).

Incubating cells with FTS produced a stable decrease in mTOR activity that persisted even when mTOR was immunoprecipitated. FIG. 12A presents results of immune complex kinases assays with AU1-mTOR from extracts of 293T cells that had been incubated with increasing concentrations of FTS. The dose response curves for FTS-mediated inhibition of AU1-mTOR activity (FIG. 12A) and dissociation of AU1-mTOR and HA-raptor (FIG. 12B) were very similar, with half maximal effects occurring between 20-30 μM. These results indicate that FTS inhibits mTOR in cells by promoting dissociation of raptor from mTORC1.

The effects of incubating cells with increasing concentrations of S-geranylthiosalicylate (GTS) on mTOR activity and the association of AU1-mTOR and HA-raptor (FIG. 12) was also investigated. GTS is identical to FTS except that it contains the 10-carbon geranyl group instead of the 15-carbon farnesyl group. GTS is much less effective than FTS in down-regulating the Ras signaling pathway, and it serves as a control for nonspecific detergent-like actions that occur with high concentrations of isoprenoid derivatives. Incubating cells with increasing concentrations of GTS slightly decreased mTOR activity (FIG. 12A); however, GTS was clearly less effective than FTS. GTS had relatively little effect on the association of AU1-mTOR with either HA-raptor or HA-mLST8 (FIG. 12B). Incubating cells with 200 μM sodium salicylate was also without effect on either mTOR activity or the association of mTOR and raptor.

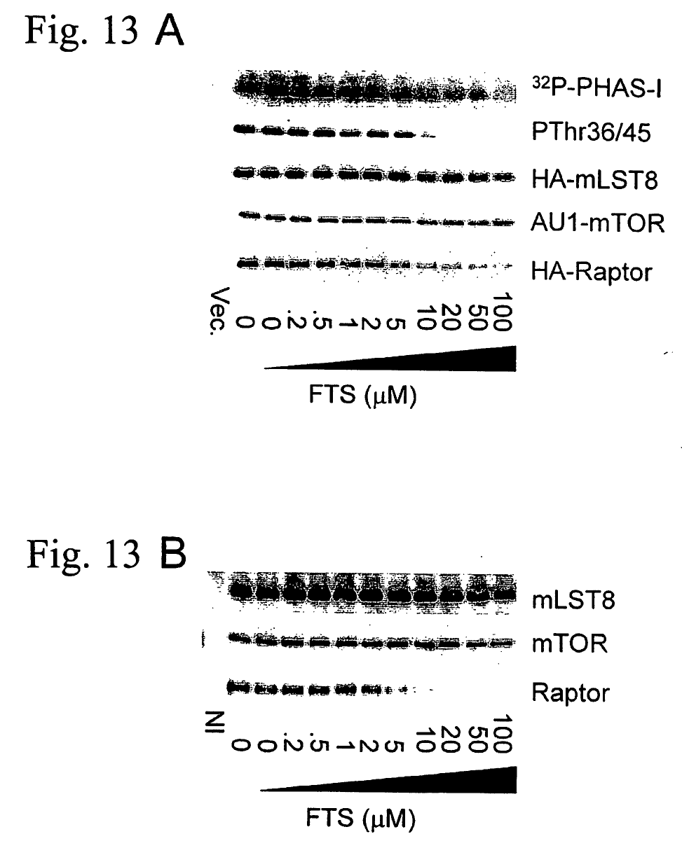

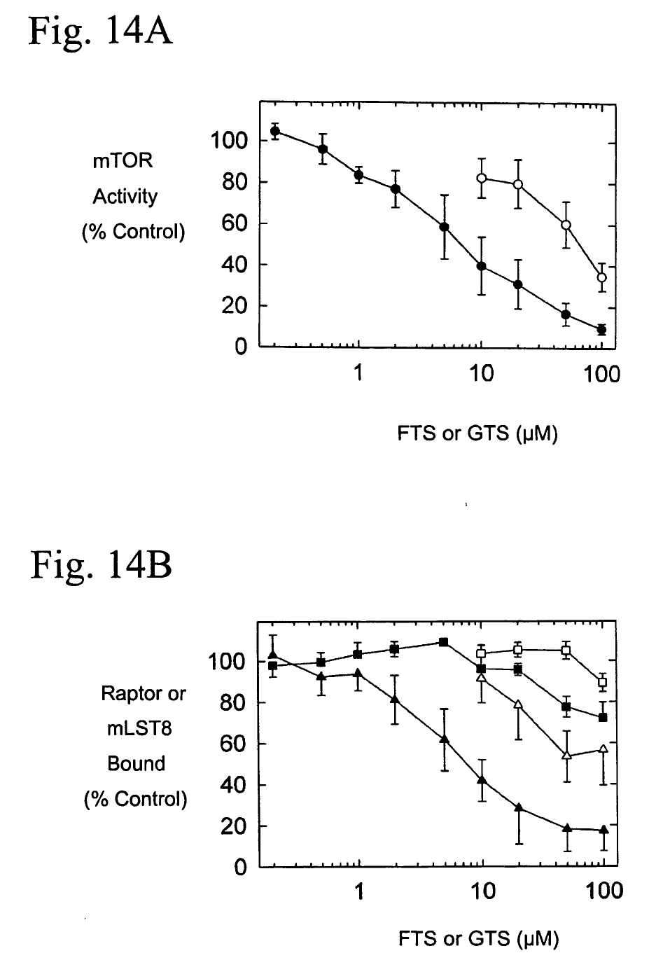

The findings with FTS in intact cells would be consistent with either an action of FTS on TORC1 or an action on a signaling pathway controlling the association of mTOR and raptor. Since the integrity of most signaling pathways is disrupted when cells are homogenized, the effects of FTS in extracts of cells in which AU1-mTOR, HA-raptor, and HA-mLST9 had been overexpressed was investigated. Incubating extracts with increasing concentrations of FTS progressively decreased the PHAS-I kinase activity of AU1-mTOR, assessed both by ³²P incorporation from [γ-³²P]ATP and by immunoblotting with PThr36/45 antibodies (FIG. 13A). Approximately four times lower concentrations of FTS were needed to inhibit mTOR activity in vitro than in intact cells. Presumably factors related to protein binding and membrane permeability account for the difference in concentrations of FTS needed in cells and extracts. Incubating extracts with increasing concentrations of FTS also decreased the amount of HA-raptor that coimmunoprecipitated with AU 1-mTOR. The dose response curves of kinase inhibition (FIG. 14A) and dissociation of AU1-mTOR and HA-raptor (FIG. 14B) were almost identical, indicating that loss of raptor from mTORC1 accounted for the inhibition of mTOR activity by FTS under these in vitro conditions. Again, the effects of FTS did not depend on overexpression of the mTORC1 components. Incubating extracts of nontransfected cells with FTS decreased the amount of endogenous raptor that coimmunoprecipitated with mTOR (FIG. 13B). The effects occurred at the same concentrations that promoted dissociation of the complex between the transfected proteins (FIG. 13A). The inhibitory effects of FTS on mTOR activity were apparent if FTS was added directly to immune complexes just before the protein kinase assay, or if FTS was added to extracts prior to the immunoprecipitation of mTOR. Thus, if FTS action depends on factors other than the known components of mTORC1, such factors must coimmunoprecipitate with the complex. FTS also modestly decreased the amounts of both transfected and endogenous mLST8 proteins that coimmunoprecipitated with mTOR proteins (FIGS. 13A and 13B), but the effects were observed only at the highest concentrations of FTS investigated (FIG. 14B).

Incubating extracts of transfected cells with increasing concentrations of GTS also led to progressive decreases both in AU1-mTOR activity (FIG. 14A) and in the association of AU1-mTOR and HA-raptor (FIG. 14B). These effects of GTS occurred at approximately ten times higher concentrations than those of FTS. Thus, selectivity is conferred by the isoprenyl component of the drug.

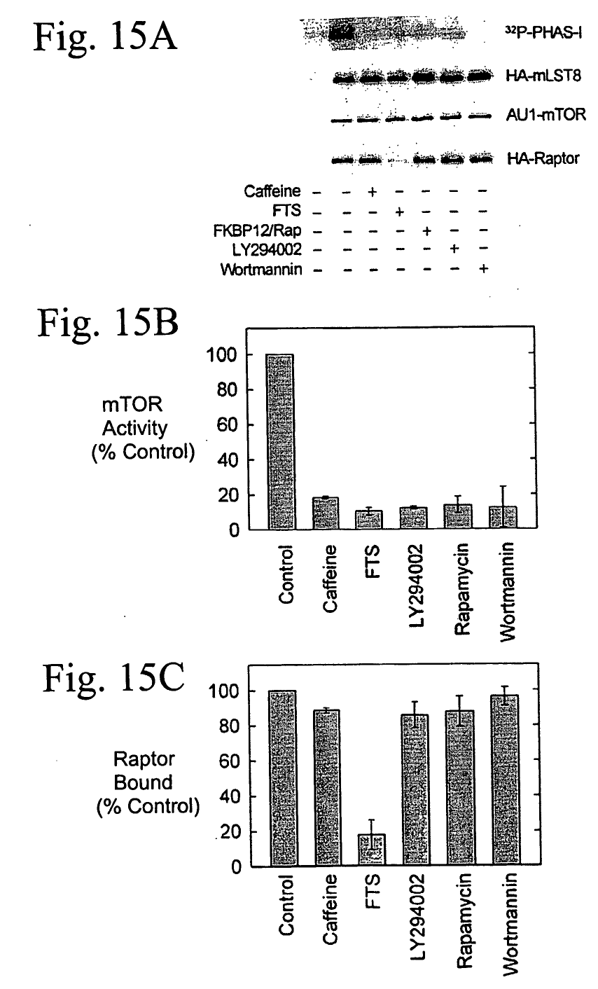

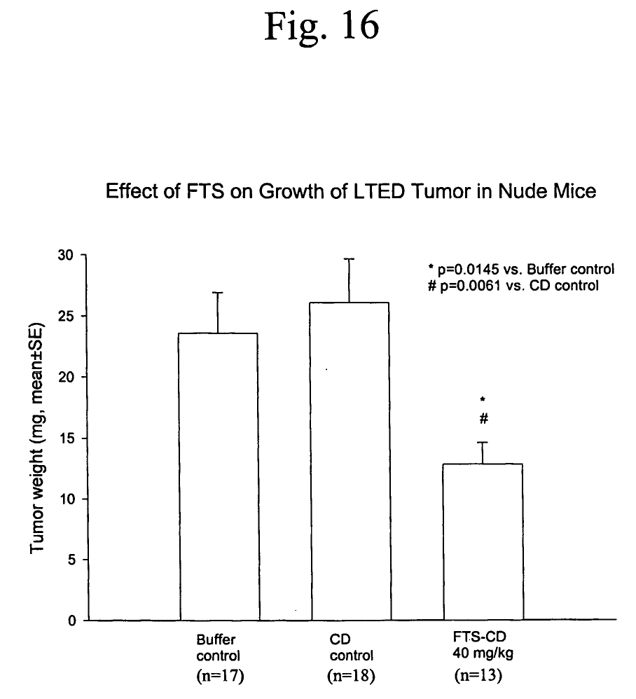

The effects of FTS to those of rapamycin and several other inhibitors of mTOR that have been described previously were also compared. Incubating extracts from transfected cells with FTS decreased both mTOR activity (FIGS. 15A and 15B) and the association of AU1-mTOR and HA-raptor (FIGS. 15A and 15C). In contrast, incubating these complexes with concentrations of caffeine, rapamycin-FKBP12, LY294002 or wortmannin that decreased mTOR activity by more than 80% had little, if any, effect on decreasing the association of AU1-mTOR and HA-raptor.

The results of this study provide direct evidence that FTS inhibits mTOR activity. This is based upon the following evidence: (1) FTS blocks PHAS-1 and P70 S6 kinase activity to a substantially greater extent than it inhibits AKT or MAP kinase. (2) direct mTOR assays using transfected HA tagged mTOR, humagglutiin precipitation of mTOR and specific substate assays demonstrate a direct effect of FTS on mTOR activity (3) FTS interferes with the binding of mTOR to RAPTOR, a step necessary for the actions of mTOR and (4) mTOR appears to be farnesylated as demonstrated indirectly with an antibody specific for farnesylated proteins. The finding that the inhibition of mTOR activity by increasing concentrations of FTS correlated closely with the dissociation of the mTOR-raptor complex, both in cells and in vitro, supports the conclusion that FTS acts by promoting dissociation of raptor from mTORC1. FTS appears to be the first example of a drug that inhibits mTOR signaling in this manner.

The peptidomimetic farnesyltransferase inhibitor, L-744,832, has also been shown to inhibit mTOR signaling. By analogy to the Ras signaling pathway, it is logical to suspect that FTS and farnesyltransferase inhibitors might act at the same target in the mTOR signaling pathway. Both FTS and farnesyltransferase inhibitors disrupt the plasma membrane localization of Ras, one by blocking in the isoprenylation of Ras necessary for its membrane localization, the other by displacing Ras from its membrane binding sites. Farnesyltransferases prenylate the Cys found in a COOH terminal motif, sometimes referred to as the CAAX box (where C is Cys, A is an aliphatic amino acid, and X is any amino acid). The COOH terminal sequence in mTOR is CysProPheTrp, which has some features of a CAAXbox. However, based on studies with model peptides, the Phe in the mTOR sequence represents a strong negative determinant. Indeed, peptides with Phe in this position served as the basis for the design of L-744,832, and other potent competitive inhibitors of farnesyltransferase.

While none of the proteins in mTORC1 are known to be prenylated, there are potential targets for FTS upstream in the mTOR signaling pathway. One example is the farnesylated GTP-binding protein Rheb (Ras homologue enriched in brain). Rheb is activated in response to growth factors that inhibit the TSC1/TSC2, which functions as the Rheb GTP'ase activating protein (GAP) (Yang, et al., (1999) FEBS Lett. 453, 387-390; Aharonson, et al., (1998) Biochim. Biophys. Acta 1406, 40-50; and Brunn, et al., (1996) EMBO J. 15, 5256-5267). Although the mechanism is still unclear, activation of Rheb increases mTOR signaling. Mutating the Cys in the CAAX box of Rheb abolished the ability of overexpressed Rheb to increase S6K activity. Thus, Rheb is a potential target for farnesyltransferase inhibitors, and it is feasible that an action of FTS to displace Rheb from intracellular binding sites contributes to the inhibitory effects of FTS on mTOR signaling in intact cells. However, Rheb does not appear to coimmunoprecipitate with mTOR. Thus, it is not clear that Rheb was involved in the inhibitory effects of FTS on mTOR activity and the association of mTOR and raptor in vitro. Interestingly, the inhibition of mTOR signaling by L-744,832 in cells seems to occur too rapidly (within 1.5 h) to be explained by inhibition of protein farnesylation.

Consistent with its action to inhibit Ras signaling, FTS blocks the activation of MAP kinase, and it inhibits the proliferation of several types of tumor cells, both in vitro and in vivo (Law, et al., (2000) J. Biol. Chem. 275, 10796-10801; Casey, P. J. and Seabra, M. C. (1996) J. Biol. Chem. 271, 5289-5292; Yamagata, et al., (1994) J. Biol. Chem. 269, 16333-16339; and Zhang, et al., (2003) Nature Cell Biol. 5, 578-581). In view of the number of different proteins that are farnesylated, the actions of FTS would be expected to involve more than inhibition of Ras signaling. As reported herein, inhibition by FTS of the effect of estrogen on stimulating the proliferation of breast cancer cells correlates much better with dephosphorylation of PHAS-I and S6K-1, two downstream elements of the mTOR signaling pathway, than with the inhibition of MAPK.

Inhibition of mTOR with rapamycin has been shown to inhibit translation of capped mRNAs and messages having a TOP (tract of pyrimidines) motif adjacent to the cap site. There are also reasons to suspect that the inhibition of mTORC1, with the decrease in PHAS-I phosphorylation and the resulting decrease in the availability of eIF4E for translation, contributes to the antiproliferative effect of FTS. Increasing eIF4E may result not only in an increase in cap-dependent translation, but also in an increase in cell proliferation. For example, overexpressing eIF4E increased the rate of growth and caused an aberrant morphology of HeLa cells. Stable overexpression of eIF4E in 3T3 fibroblasts not only increased the rate of proliferation but actually caused malignant transformation, as evidenced by anchorage independent growth and formation of tumors when implanted in nude mice. eIF4E levels are elevated in the majority of breast cancers, which also frequently contain mutant forms of Ras (Jansen, et al., (1999) J. Mol. Med. 77, 792-797).

It has been suggested that eIF4E stimulates proliferation by preferentially increasing translation of proteins that facilitate mitogenesis. The 5′-UTRs of mRNAs encoding many oncogenes, growth factors and signal transduction proteins are predicted to contain regions of relatively stable secondary structure. These structured regions have been shown to interfere with binding and/or scanning by the 40S ribosomal subunit. Translation of such messages appears to be more dependent on eIF4E availability than translation of mRNAs having unstructured 5′UTRs, a characteristic of many messages encoding house-keeping proteins. The dependency on eIF4E is believed to be explained by the requirement of eIF4E for formation of eIF4F, which melts secondary structure in the 5′-UTR via the helicase activity of the eIF4A subunit. As predicted from this mechanism, overexpressing PHAS-I, which decreases eIF4E availability, caused reversion of cells overexpressing eIF4E. Interestingly, overexpressing a constitutively active PHAS-I protein was recently shown to decreased the proliferation of MCF7 breast cancer cells (Terada, et al., (1994) Proc. Natl. Acad. Sci. U.S.A. 91, 11477-11481).

By inhibiting mTOR activity and decreasing PHAS-I phosphorylation, FTS should decrease the contribution of eIF4E to proliferative responses. Other mTOR-dependent processes that are independent of changes in eIF4E availability are surely involved in the control of cell proliferation. It is anticipated that FTS will be found to inhibit those processes requiring the raptor-mTOR interaction. Accordingly one embodiment of the present invention is directed to the ability to block mTOR activity with FTS. This effect can be put to use for the treatment of breast cancer. In this disease, FTS will delay the development of resistance to estrogen deprivation therapy.

Materials and Methods

Antibodies—Antibodies recognizing endogenous mTOR (mTAb1 and mTAb2), PHAS-I, and raptor were generated by immunizing rabbits with peptides having sequences corresponding to regions in the respective proteins. The phosphospecific antibodies, P-Thr36/45 and P-Thr69, that recognize phosphorylated sites in PHAS-I were generated as described previously (Mothe-Satney et al., (2000) J. Biol. Chem. 275, 33836-33843). P-Thr36/45 antibodies bind to PHAS-I phosphorylated in either Thr36 or Thr45, as the sequences surrounding these sites are almost identical. Ascites fluid containing monoclonal antibody to the AU1 epitope tag was from Berkley Antibody Company. 9E10, which recognizes the myc epitope tag, and 12CA5, which recognizes the HA epitope tag, were purified from hybridoma culture medium by the University of Virginia Lymphocyte Culture Center.

To generate antibodies to mLST8, a synthetic peptide having a sequence identical to positions 298-313 of human mLST8 was coupled to keyhole limpet hemocyanin, and the conjugate was used to immunize rabbits as described previously (Zimmer, et al., (2000) Anticancer Res 20, 1343-¹³⁵I). Antibodies were purified using a column containing affinity resin prepared by coupling the peptide to Sulfolink beads (Pierce).

cDNA Constructs—The pcDNA3^{AU1-mTOR}, pcDNA3^{3HA-Raptor}, and pCMV-Tag 3A^{PHAS-I constructs for overexpressing AU}1-mTOR, HA-Raptor, and myc-PHAS-I were described previously (Brunn, et al., (1997) Science 277, 99-101; Choi, et al., (2003) J. Biol. Chem. 276, 19667-19673; and Kozak, M. (1991) J. Cell Biol. 115, 887-903). pcDNA3^{3HA-mLST8} encodes mLST8 having an NH₂ terminal triple HA epitope tag (HA-mLST8). To generate pcDNA3^{3HA-mLST8} a 5′ EcoRI site and a 3′NotI site were introduced into mLST8 cDNA by PCR using I.M.A.G.E. clone 3910883 as template. After digesting the product with EcoRI and NotI, the mLST8 cDNA was inserted in pcDNA3^{3HA-Raptor} in place of raptor insert, which had been removed with EcoRI and NotI. The coding region of the resulting pcDNA3^{3HAmLST8} was sequenced and found to be free of errors.

Overexpression of AU1-mTOR, HA-raptor, HA-mLST8 and Myc-PHAS-I-293T cells were cultured for 24 h in growth medium composed of 10% (v/v) fetal bovine serum in Dulbecco's modified Eagle medium (DMEM). AU1-mTOR, HA-raptor, and HA-mLST8 were coexpressed by transfecting 293T cells (100 mm diameter dish) with 4 μg each of pcDNA3^{AU1-mTOR}, pcDNA3^{3HA-Raptor} and pcDNA3^{3HA-mLsT8} by using TransIT-LT2 (Mirus Corp., Madison, Wis.) as described previously (Brunn, et al., (1997) Science 277, 99-101). Other cells were transfected with pcDNA3 vector alone. Where indicated, cells were transfected with pCMV-Tag 3A^{PHAS-I} to coexpress Myc-PHAS-I. Cells were used in experiments 18-20 hours after transfection.

Immune complex assay of mTOR activity—AU1-mTOR was immunoprecipitated by using anti-AU1 antibody bound to protein G-agarose beads as described previously (McMahon, et al., (2002) Mol. Cell. Biol. 22, 7428-7438). Endogenous mTOR was immunoprecipitated in the same manner, except that mTAb1 was used instead of anti-AU1 antibody. To measure kinase activity, exhaustively washed immune complexes were suspended in 20 μl of Buffer A (50 mM NaCl, 0.1 mM EGTA, 1 mM dithiothreitol (DTT), 0.5 mM microcystin LR, 10 mM Na-HEPES, and 50 mM β-glycerophosphate, pH 7.4.). The kinase reactions were initiated by adding 20 μl of Buffer A supplemented with 2 mM [γ-³²P]ATP, 20 mM MnCl₂ and 40 μg/ml of [His⁶]PHAS-I. In experiments in which the effects of wortmannin were investigated, DTT was omitted from the reactions, and [His⁶]PHAS-I that had been reduced and alkylated with N-ethylmaleimide was used as substrate. Reactions were terminated after 10 min at 30° by adding SDS sample buffer. Samples were subjected to SDS-PAGE and the relative amounts of ³²P incorporated into [His⁶]PHAS-I were determined.

Electrophoretic analyses—SDS-PAGE was performed using the method of Laemmli. Immunoblots were prepared after electrophoretically transferring protein to Immobilon (Millipore) membranes. The relative amounts of ³²P incorporated into [His⁶]PHAS-I were determined by phosphorimaging. Signal intensities of bands in immunoblots were determined by scanning laser densitometry.

Other materials—FTS and S-geranylthiosalicylic acid (GTS) were kindly provided by from Dr. Yoel Kloog (Tel-Aviv University, Tel-Aviv, Israel) and Wayne Bardin (Thyreos, New York, N.Y.). Rapamycin and LY294002 were from Calbiochem-Novabiochem International. Caffeine was from Sigma Chemical Co. Glutathione S transferase (GST)-FKBP12 and [His⁶]PHAS-I were expressed in bacteria and purified as described previously (Rousseau, et al. (1996) Oncogene 13, 2415-2420; Jiang, et al., (2003) Cancer Cell Int. 3, 2). [γ-³²P]ATP was from NEN Life Science Products.

1. A composition for treating hormone responsive cancers, said composition comprising an mTOR inhibitor of the general structure: 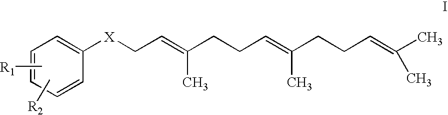
wherein X is NH, O or S; R₁ is H or halo; and R₂ is COOH and a compound selected from the group consisting of estrogen antagonists and aromatase inhibitors.

2. The composition of claim 1 wherein X is O.

3. The composition of claim 1 wherein X is S.

4. The composition of claim 1 wherein X is NH.

5. The composition of any of claims 2-4 wherein R₁ is H.

6. The composition of any of claims 2-4 wherein R₁ is F.

7. The composition of claim 1 wherein the mTOR inhibitor has the general structure: 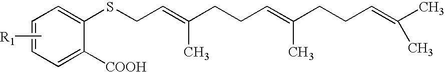
wherein R₁ is H or halo.

8. The composition of claim 7 wherein R₁ is H.

9. The composition of claim 7 wherein R₁ is Cl.

10. The composition of claim 7 wherein R₁ is F.

11. The composition of claim 5, 8, 9 or 10 wherein the estrogen antagonist is tamoxifin.

12. The composition of claim 5, 8, 9 or 10 wherein the aromatase inhibitor is selected from the group consisting of exemestane, anastrozole and letrozole.

13. A method of inhibiting mTOR activity in a cell, said method comprising the step of contacting the cell with a composition comprising a compound of the general structure: 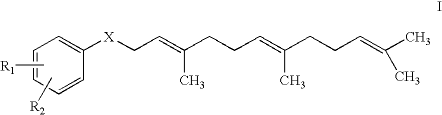
wherein X is NH, O or S; R₁ is H or halo; and R₂ is COOH.

14. The method of claim 13 wherein the inhibitory effect results from the dissociation of raptor from mTOR.

15. The method of claim 13 wherein the inhibitory effect does not inhibit activation of Akt.

16. The method of claim 14 wherein X is O, and R₁ is H or F.

17. The method of claim 14 wherein X is S and R₁ is H or F.

18. The method of claim 16 or 17 wherein R₁ is H.

19. A method of inhibiting the growth of an adapted hormone responsive cancer cell said method comprising the step of administering a compound of the general formula: 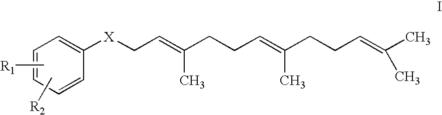
wherein X is NH, O or S; R₁ is H or halo; and R₂ is COOH.

20. The method of claim 19 wherein the hormone responsive cancer cell is an estrogen responsive cancer cell.

21. The method of claim 20 wherein the estrogen responsive cancer cell is a breast cancer cell.

22. The method of claim 20 or 21 wherein X is O and R₁ is H or F.

23. A method for slowing or preventing the progression of the adaptive response in a patient, said method comprising the steps of administering to said patient a compound of the general structure: 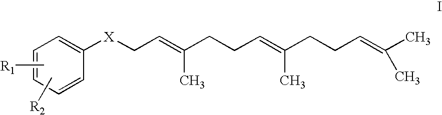
wherein X is NH, O or S; R₁ is H or halo; and R₂ is COOH.

24. The method of claim 23 wherein the compound of formula I is administered to the patient in conjunction with the administration of hormone deprivation therapy.

25. The method of claim 24 wherein the hormone deprivation therapy comprises the administration of an estrogen antagonist.

26. The method of claim 25 wherein the estrogen antagonist is tamoxifen.

27. The method of claim 25 wherein the hormone deprivation therapy comprises the administration of an aromatase inhibitor.

28. A method of enhancing the rate of apoptosis in a population of hormone responsive cancer cells, said method comprising the step of administering a compound of the general structure: 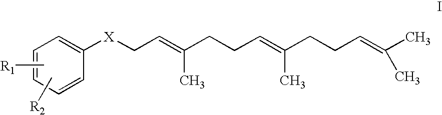
wherein X is NH, O or S; R₁ is H or halo; and R₂ is COOH.

29. The method of claim 28 wherein the hormone responsive cancer cells are responsive to estrogen.

30. The method of claim 29 wherein the hormone responsive cancer cells are breast cancer cells.

31. The method of claim 29 or 30 wherein X is S and R₁ is H or F.

32. A method of treating an estrogen responsive breast or ovarian cancer, said method comprising the step of administering a compound of the general structure: 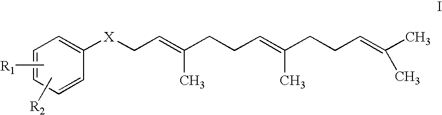
wherein X is NH, O or S; R₁ is H or halo; and R₂ is COOH.

33. The method of claim 32 wherein X is S and R₁ is H or F.
